SEQUENCE LISTING (3 sequences)

SEQ ID NO: 1 - protein, 6 aa, Homo sapiens
MELADL

SEQ ID NO: 2 - DNA, 13592 bp, synthetic construct
  source 1..13592: mol_type = other DNA
atcgtacaagaaagctgggtctagtcgacaggcgcgcccccagggatgtaattacgtccctaacccgctagggggcagcaggcgcgcctctcgacaggcgcgcccccagggatgtaattacgtccctaacccgctagggggcagcaggcgcgcctctcgcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtacctgagctcgctagcctcgaggatcgataggtactcggaggacagtactccgtactcggaggacagtactccgtactcggaggacagtactccgtactcggaggacagtactccgtacctcggaggacagtactccgctcggaggacagtactccgctcggaggacagtactccgctcggaggacagtactccgatccgtcgactctagagggtatataagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatcaaatgatatcgtacaagaaagctgggtctagtcgacaggcgcgcccccagggatgtaattacgtccctaacccgctagggggcagcaggcgcgcctctcgacaggcgcgcccccagggatgtaattacgtccctaacccgctagggggcagcaggcgcgcctctcgcggccgcgaattcggtaccggatcacgcgtgctagcccgggctcgagatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaaagcttgattcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagccaccatgacttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagaccttattggtatgggcaaatcaggcaaatctggtaatggttcttataggttacttgatcattacaaatatcttactgcatggtttgaacttcttaatttaccaaagaagatcatttttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggacccaggattcttttccaatgctattgttgaaggtgccaagaagtttcctaatactgaatttgtcaaagtaaaaggtcttcatttttcgcaagaagatgcacctgatgaaatgggaaaatatatcaaatcgttcgttgagcgagttctcaaaaatgaacaataattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgaattcactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttgtcgacgccaccatggaacaaaaactcatctcagaagaggatctgatgaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttaacaggattatttgtacaagataatgtgaataaagatgccgtcacagatagattggcttcagtggagactgatatgcctctaacattgagacagcatagaataagtgcgacatcatcatcggaagagagtagtaacaaaggtcaaagacagttgactgtatcgccggaattcccagccgacgcccttgacgattttgaccttgacatgctccctgcagatgcccttgacgactttgaccttgatatgctgcctgctgacgctcttgacgattttgaccttgacatgctcgaaggatccttcgaccaggccaaatcggccatggattgcactttcgaagacatgcttcagcttatcaacaaccaagacagtgacttccctggcctatttgacccaccctatgctgggagtggggcagggggcacagaccctgccagccccgataccagctccccaggcagcttgtctccacctcctgccacattgagctcctctcttgaagccttcctgagcgggccgcaggcagcgccctcacccctgtcccctccccagcctgcacccactccattgaagatgtacccgtccatgcccgctttctcccctgggcctggtatcaaggaagagtcagtgccactgagcatcctgcagacccccaccccacagcccctgccaggggccctcctgccacagagcttcccagccccagccccaccgcagttcagctccacccctgtgttaggctaccccagccctccgggaggcttctctacaggaagccctcccgggaacacccagcagccgctgcctggcctgccactggcttccccgccaggggtcccgcccgtctccttgcacacccaggtccagagtgtggtcccccagcagctactgacagtcacagctgcccccacggcagcccctgtaacgaccactgtgacctcgcagatccagcaggtcccggtcctgctgcagccccacttcatcaaggcagactcgctgcttctgacagccatgaagacagacggagccactgtgaaggcggcaggtctcagtcccctggtctctggcaccactgtgcagacagggcctttgccgaccctggtgagtggcggaaccatcttggcaacagtcccactggtcgtagatgcggagaagctgcctatcaaccggctcgcagctggcagcaaggccccggcctctgcccagagccgtggagagaagcgcacagcccacaacgccattgagaagcgctaccgctcctccatcaatgacaaaatcattgagctcaaggatctggtggtgggcactgaggcaaagctgaataaatctgctgtcttgcgcaaggccatcgactacattcgctttctgcaacacagcaaccagaaactcaagcaggagaacctaagtctgcgcactgctgtccacaaaagcaaatctctgaaggatctggtgtcggcctgtggcagtggagggaacacagacgtgctcatggagggcgtgaagactgaggtggaggacacactgaccccacccccctcggatgctggctcacctttccagagcagccccttgtcccttggcagcaggggcagtggcagcggtggcagtggcagtgactcggagcctgacagcccagtctttgaggacagcaaggcaaagccagagcagcggccgtctctgcacagccggggcatgctggaccgctcccgcctggccctgtgcacgctcgtcttcctctgcctgtcctgcaaccccttggcctccttgctgggggcccgggggcttcccagcccctcagataccaccagcgtctaccatagccctgggcgcaacgtgctgggcaccgagagcagagatggccctggctgggcccagtggctgctgcccccagtggtctggctgctcaatgggctgttggtgctcgtctccttggtgcttctctttgtctacggtgagccagtcacacggccccactcaggccccgccgtgtacttctggaggcatcgcaagcaggctgacctggacctggcccggggagactttgcccaggctgcccagcagctgtggctggccctgcgggcactgggccggcccctgcccacctcccacctggacctggcttgtagcctcctctggaacctcatccgtcacctgctgcagcgtctctgggtgggccgctggctggcaggccgggcagggggcctgcagcaggactgtgctctgcgagtggatgctagcgccagcgcccgagacgcagccctggtctaccataagctgcaccagctgcacaccatggggaagcacacaggcgggcacctcactgccaccaacctggcgctgagtgccctgaacctggcagagtgtgcaggggatgccgtgtctgtggcgacgctggccgagatctatgtggcggctgcattgagagtgaagaccagtctcccacgggccttgcattttctgacacgcttcttcctgagcagtgcccgccaggcctgcctggcacagagtggctcagtgcctcctgccatgcagtggctctgccaccccgtgggccaccgtttcttcgtggatggggactggtccgtgctcagtaccccatgggagagcctgtacagcttggccgggaacccagtggaccccctggcccaggtgactcagctattccgggaacatctcttagagcgagcactgaactgtgtgacccagcccaaccccagccctgggtcagctgatggggacaaggaattctcggatgccctcgggtacctgcagctgctgaacagctgttctgatgctgcgggggctcctgcctacagcttctccatcagttccagcatggccaccaccaccggcgtagacccggtggccaagtggtgggcctctctgacagctgtggtgatccactggctgcggcgggatgaggaggcggctgagcggctgtgcccgctggtggagcacctgccccgggtgctgcaggagtctgagagacccctgcccagggcagctctgcactccttcaaggctgcccgggccctgctgggctgtgccaaggcagagtctggtccagccagcctgaccatctgtgagaaggccagtgggtacctgcaggacagcctggctaccacaccagccagcagctccattgacaaggccgtgcagctgttcctgtgtgacctgcttcttgtggtgcgcaccagcctgtggcggcagcagcagcccccggccccggccccagcagcccagggcaccagcagcaggccccaggcttccgcccttgagctgcgtggcttccaacgggacctgagcagcctgaggcggctggcacagagcttccggcccgccatgcggagggtgttcctacatgaggccacggcccggctgatggcgggggccagccccacacggacacaccagctcctcgaccgcagtctgaggcggcgggcaggccccggtggcaaaggaggcgcggtggcggagctggagccgcggcccacgcggcgggagcacgcggaggccttgctgctggcctcctgctacctgccccccggcttcctgtcggcgcccgggcagcgcgtgggcatgctggctgaggcggcgcgcacactcgagaagcttggcgatcgccggctgctgcacgactgtcagcagatgctcatgcgcctgggcggtgggaccactgtcacttccagctagggccataagggcctctagagggcccgtttaaacccgctgatcagcctcgactgtgccttctagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaat

SEQ ID NO: 3 - DNA, 13607 bp, synthetic construct
  source 1..13607: mol_type = other DNA
atcgtacaagaaagctgggtctagtcgacaggcgcgcccccagggatgtaattacgtccctaacccgctagggggcagcaggcgcgcctctcgacaggcgcgcccccagggatgtaattacgtccctaacccgctagggggcagcaggcgcgcctctcgcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtacctgagctcgctagcctcgaggatcgataggtactcggaggacagtactccgtactcggaggacagtactccgtactcggaggacagtactccgtactcggaggacagtactccgtacctcggaggacagtactccgctcggaggacagtactccgctcggaggacagtactccgctcggaggacagtactccgatccgtcgactctagagggtatataagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatcaaatgatatcgtacaagaaagctgggtctagtcgacaggcgcgcccccagggatgtaattacgtccctaacccgctagggggcagcaggcgcgcctctcgacaggcgcgcccccagggatgtaattacgtccctaacccgctagggggcagcaggcgcgcctctcgcggccgcgaattcggtaccggatcacgcgtgctagcccgggctcgagatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaaagcttgattcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagccaccatgacttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagaccttattggtatgggcaaatcaggcaaatctggtaatggttcttataggttacttgatcattacaaatatcttactgcatggtttgaacttcttaatttaccaaagaagatcatttttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggacccaggattcttttccaatgctattgttgaaggtgccaagaagtttcctaatactgaatttgtcaaagtaaaaggtcttcatttttcgcaagaagatgcacctgatgaaatgggaaaatatatcaaatcgttcgttgagcgagttctcaaaaatgaacaataattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgaattcactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttgtcgacgccaccatggaacaaaaactcatctcagaagaggatctgatgaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttaacaggattatttgtacaagataatgtgaataaagatgccgtcacagatagattggcttcagtggagactgatatgcctctaacattgagacagcatagaataagtgcgacatcatcatcggaagagagtagtaacaaaggtcaaagacagttgactgtatcgccggaattcccagccgacgcccttgacgattttgaccttgacatgctccctgcagatgcccttgacgactttgaccttgatatgctgcctgctgacgctcttgacgattttgaccttgacatgctcgaaggatccttcgaccaggccaaatcggccgagaccctcacggagctgggcgacgagctgaccctgggagacatcgacgagatgctgcaatttgtcagtaatcaagtgggagagttccctgacttgttttcagaacagctgtgtagctcctttcctggcagtggtggtagtggtagcagcagcggcagcagtggcagcagcagcagcagcagcaatggcaggggcagcagcagcggagctgtggacccttcagtgcaacggtcattcacccaggtcacattaccttccttctctccctcggcggcctccccacaggctccaactctgcaagtcaaggtttctcccacctcagttcccaccacacccagggcaactcctattcttcagccccgcccccagccccagcctcaacctcaaactcagctgcaacaacagacggtaatgatcacgccaacattcagcaccactccgcagacgaggatcatccagcagcctttgatataccagaatgcagctactagctttcaagtccttcagcctcaagtccaaagcctggtgacatcctcccaggtacagccggtcaccattcagcagcaggtgcagacagtacaggcccagcgggtgctgacacaaacggccaatggcacgctgcagacccttgccccggctacggtgcagacagttgctgcgccacaggtgcagcaggtcccggtcctggtccagcctcagatcatcaagacagattcccttgttttgaccacactgaagacagatggcagccctgttatggctgcggtccagaacccggccctcaccgccctcaccacccctatccagacggctgcccttcaagtaccaaccctggtgggcagcagtgggaccattctgaccacaatgcctgtaatgatggggcaagagaaagtgcccattaagcaggtacctgggggagtcaagcagcttgagccccccaaagaaggagaaaggcggacaacccataatatcattgagaaacgatatcgctcctccatcaatgacaaaatcatcgaattgaaagacctggtcatggggacagacgccaagatgcacaagtctggcgttctgaggaaggccattgattacatcaaatacttgcagcaggtcaatcataaactgcgccaggagaacatggtgctgaagctggcaaatcaaaagaacaagcttctaaagggcatcgacctaggcagtctggtggacaatgaggtggacctgaagatcgaggactttaatcagaatgtccttctgatgtcccccccagcctctgactcagggtcccaggctggcttctctccctactccattgactctgagccaggaagccctctattggatgatgcaaaggtcaaagatgagccagactctcctcctgtggcgctgggcatggtagaccgctcacggattcttctgtgtgtcctcaccttcctgtgcctctcctttaaccccctgacttccctgctgcagtggggaggggcccacgactctgaccagcacccacactcaggctctggccgcagtgtcctgtcattcgagtcaggttctgggggctggtttgactggatgatgcctactcttctcttatggctggtaaatggtgtgattgtcctgagcgtctttgtgaagctgctggttcatggggagccagtgatccggccacactcgcgctcctcggtcaccttctggaggcaccggaaacaggcagatctggatctcgccagaggagattttgcagctgctgccggcaacctacaaacctgcctggcagttttgggccgggcactgcccacctcccgcctggacctggcctgcagcctctcctggaacgtgatccgctacagcctgcagaagctacgcctggtgcgctggctgctcaagaaagtcttccagtgccggcgggccacgccagccactgaggcaggctttgaagacgaagctaagaccagcgcccgggatgcggctctggcctatcaccggctgcaccagctgcacatcacagggaagcttcctgcaggatccgcctgttccgatgtacacatggcgttgtgtgccgtgaacctggctgaatgtgcagaggagaagatcccaccgagcacactggttgagatccatctgactgctgccatggggctcaagacccggtgtggaggcaagctgggcttcctggccagctacttcctcagccgagcccagagcctgtgtggccccgagcacagtgctgttcctgactccctgcgctggctctgccaccccctgggccagaagtttttcatggagcggagctggtctgtgaagtcagctgccaaggagagtctatactgtgcccagaggaacccagctgaccccattgcgcaggtccaccaggccttctgcaagaacctgctggagcgagctatagagtccttggtgaaacctcaggccaagaagaaggctggagaccaggaagaagagagctgtgaattctccagtgctctggagtacttgaaattacttcattcttttgtggactctgtgggggttatgagccccccactctccaggagctccgtgctcaagtccgccctgggtccagacatcatctgtcggtggtggacgtctgcaatcactgtggccatcagctggctccagggagacgatgcagctgtgcgctctcattttaccaaagtggaacgcatccccaaggccctggaagtgacagagagccccctggtgaaggccatcttccatgcctgcagagccatgcatgcctcactccctgggaaagcagatgggcagcagagttccttctgccattgcgagagggccagtggccacctatggagcagcctcaacgtcagtggggccacctctgaccctgccctcaaccacgtggtccagctgctcacctgtgacctgctactgtcgctacggacagcgctctggcaaaaacaggccagtgccagccaggctgtgggggagacctaccacgcgtcaggcgctgaactggcgggcttccaacgggacctgggcagcctgcgcaggctggcacacagcttccgcccagcataccgcaaggtgttcctgcatgaagccaccgtgcgcctgatggcaggagccagccccacccgcacccaccagctgctggaacacagcctgcggcggcgcaccacgcagagcaccaagcacggagaggtggatgcctggcccggccagcgagagcgggccaccgccatcctgctggcctgccgccacctgcccctctccttcctctcctccccgggccagcgggcagtgctgctggccgaagctgcccgcaccctggagaaggtgggcgaccggcgctcctgcaacgactgccagcagatgattgttaagctgggtggtggcactgccattgccgcctcctgaggccataagggcctctagagggcccgtttaaacccgctgatcagcctcgactgtgccttctagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaat

(19) United States
(12) Patent Application Publication
(10) Pub. No.: US 2025/0082612 A1
(43) Pub. Date: Mar. 13, 2025

(54) SREBP-1 INHIBITOR AND PHARMACEUTICAL COMPOSITION FOR TREATING HYPERTRIGLYCERIDEMIA

(71) Applicant: Jichi Medical University, Tokyo (JP)

(72) Inventors: Naoya YAHAGI, Shimotsuke-shi (JP); Yoshinori TAKEUCHI, Shimotsuke-shi (JP)

(21) Appl. No.: 18/721,735

(22) PCT Filed: Dec. 19, 2022
(86) PCT No.: PCT/JP2022/046579

§ 371 Date: Jun. 19, 2024

(30) Foreign Application Priority Data
Dec. 20, 2021 (JP) 2021-206008

(51) Int. Cl. A61K 31/426; A61K 31/341; A61K 31/381; A61K 31/4025; A61K 31/403; A61K 31/415; A61K 31/4164; A61K 31/4178; A61K 31/4245; A61K 31/427; A61K 31/4365; A61K 31/437; A61K 31/444; A61K 31/4535; A61K 31/4709; A61K 31/4745; A61K 31/506; A61K 31/513; A61K 31/519; A61K 31/52; A61K 31/538

(52) U.S. Cl. CPC A61K 31/426; A61K 31/341; A61K 31/381; A61K 31/4025; A61K 31/403; A61K 31/415; A61K 31/4164; A61K 31/4178; A61K 31/4245; A61K 31/427; A61K 31/4365; A61K 31/437; A61K 31/444; A61K 31/4535; A61K 31/4709; A61K 31/4745; A61K 31/506; A61K 31/513; A61K 31/519; A61K 31/52; A61K 31/538

(57) ABSTRACT

There is provided an SREBP-1 inhibitor that has inhibitory activity against SREBP-1 and no inhibitory activity against SREBP-2, the SREBP-1 inhibitor containing, as an active ingredient, any one or more compounds among compounds represented by Formulae (1) to (23). There is also provided a pharmaceutical composition for treating hypertriglyceridemia, which contains the SREBP-1 inhibitor and a pharmaceutically acceptable carrier.

### TECHNICAL FIELD

The present invention relates to an SREBP-1 inhibitor and a pharmaceutical composition for treating hypertriglyceridemia.

Priority is claimed on Japanese Patent Application No. 2021-206008, filed Dec. 20, 2021, the content of which is incorporated herein by reference.

### BACKGROUND ART

Hyperlipidemia in which a triglyceride level is 150 mg/dL or more in a fasting state is referred to as hypertriglyceridemia. The inventors of the present invention have previously reported that eicosapentaenoic acid (EPA), an @-3 fatty acid known as a therapeutic drug for hypertriglyceridemia, specifically inhibits a transcription factor, sterol regulatory element-binding protein-1 (SREBP-1), in the liver (see, for example, Non-Patent Documents 1 and 2). Subsequently, the inventors of the present invention have reported that in SREBP-1 knockout mice, the blood triglyceride level decreases (for example, see Non-Patent Document 3), and fatty liver (for example, see Non-Patent Document 4) or arteriosclerosis (for example, see Non-Patent Document 5 and the like) is also ameliorated.

### CITATION LIST

### Non-Patent Documents

[Non-Patent Document 1] Yahagi N et al., “A Crucial Role of Sterol Regulatory Element-binding Protein-1 in the Regulation of Lipogenic Gene Expression by Polyunsaturated Fatty Acids,” J Biol Chem., Vol. 274, No. 50, pp. 35840-35844, 1999. [Non-Patent Document 2] Takeuchi Y et al., “Polyunsaturated Fatty Acids Selectively Suppress Sterol Regulatory Element-binding Protein-1 through Proteolytic Processing and Autoloop Regulatory Circuit,” J Biol Chem., Vol. 285, No. 15, pp. 11681-11691, 2010.
[Non-Patent Document 3] Shimano H et al., “Sterol Regulatory Element-binding Protein-1 as a Key Transcription Factor for Nutritional Induction of Lipogenic Enzyme Genes,” J Biol Chem., Vol. 274, No. 50, pp. 35832-35839, 1999.
[Non-Patent Document 4] Yahagi N et al., “Absence of Sterol Regulatory Element-binding Protein-1 (SREBP-1) Ameliorates Fatty Livers but Not Obesity or Insulin Resistance in Lepob/Lepob Mice,” J Biol Chem., Vol. 277, No. 22, pp. 19353-19357, 2002.
[Non-Patent Document 5] Karasawa T et al., “Sterol Regulatory Element-Binding Protein-1 Determines Plasma Remnant Lipoproteins and Accelerates Atherosclerosis in Low-Density Lipoprotein Receptor-Deficient Mice,” ATVB, Vol. 31, No. 8, pp. 1788-1795, 2011.
[Non-Patent Document 6] Lombardi M et al., “Omega-3 fatty acids supplementation and risk of atrial fibrillation: an updated meta-analysis of randomized controlled trials,” European Heart Journal-Cardiovascular Pharmacotherapy, Vol. 7, Issue 4, pp. e69-e70, 2021.

### SUMMARY OF INVENTION

### Technical Problem

However, EPA is a fatty acid and is thus easily degraded in the body and is susceptible to degeneration such as oxidation. Therefore, the pharmacological effect is weak, and in addition, a wide range of actions on things other than SREBP-1 is known as a problem. In addition, as described in Non-Patent Document 6, EPA has also been alerted to pay attention to the side effects of atrial fibrillation.

The present invention has been made in consideration of the above circumstances, and the present invention provides an SREBP-1 inhibitor that specifically and selectively acts on SREBP-1, and a pharmaceutical composition for treating hypertriglyceridemia, which contains SREBP-1 inhibitors.

### Solution to Problem

That is, the present invention includes the following aspects.

<1> An SREBP-1 inhibitor that has inhibitory activity against SREBP-1 and has no inhibitory activity against SREBP-2, such as the SREBP-1 inhibitor containing, as an active ingredient, any one or more compounds among compounds represented by Formulae (1) to (23).

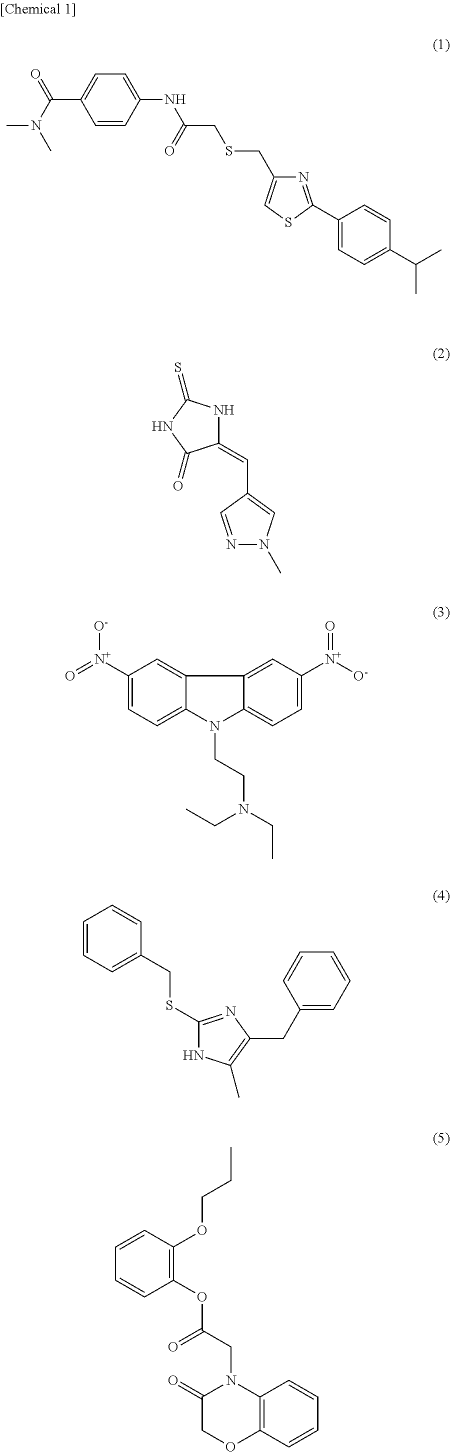 [SMILES: CC(C)c1ccc(-c2nc(CSCC(=O)Nc3ccc(C(=O)N(C)C)cc3)cs2)cc1 ; Cn1cc(/C=C2/NC(=S)NC2=O)cn1 ; CCN(CC)CCn1c2ccc([N+](=O)[O-])cc2c2cc([N+](=O)[O-])ccc21 ; Cc1[nH]c(SCc2ccccc2)nc1Cc1ccccc1 ; CCCOc1ccccc1OC(=O)CN1C(=O)COc2ccccc21]

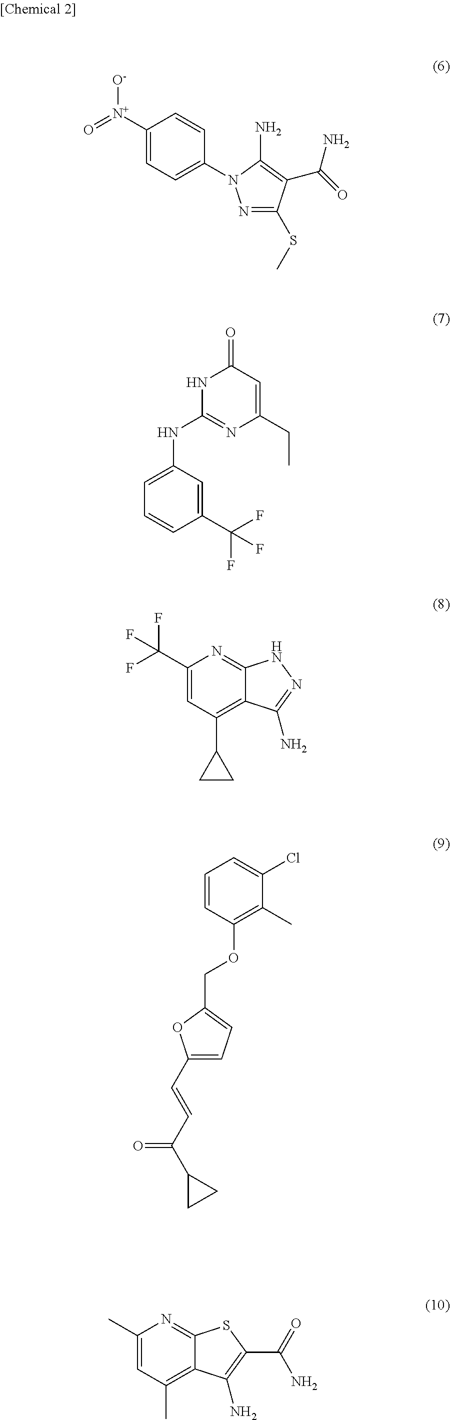

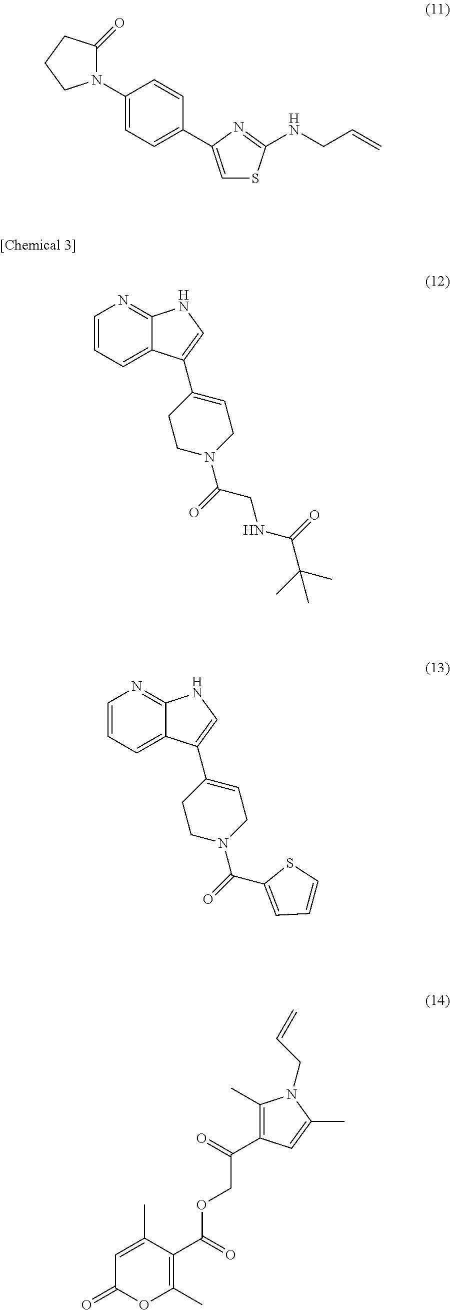 [SMILES: C=CCNc1nc(-c2ccc(N3CCCC3=O)cc2)cs1 ; CC(C)(C)C(=O)NCC(=O)N1CC=C(c2c[nH]c3ncccc23)CC1 ; O=C(c1cccs1)N1CC=C(c2c[nH]c3ncccc23)CC1 ; C=CCn1c(C)cc(C(=O)COC(=O)c2c(C)cc(=O)oc2C)c1C]

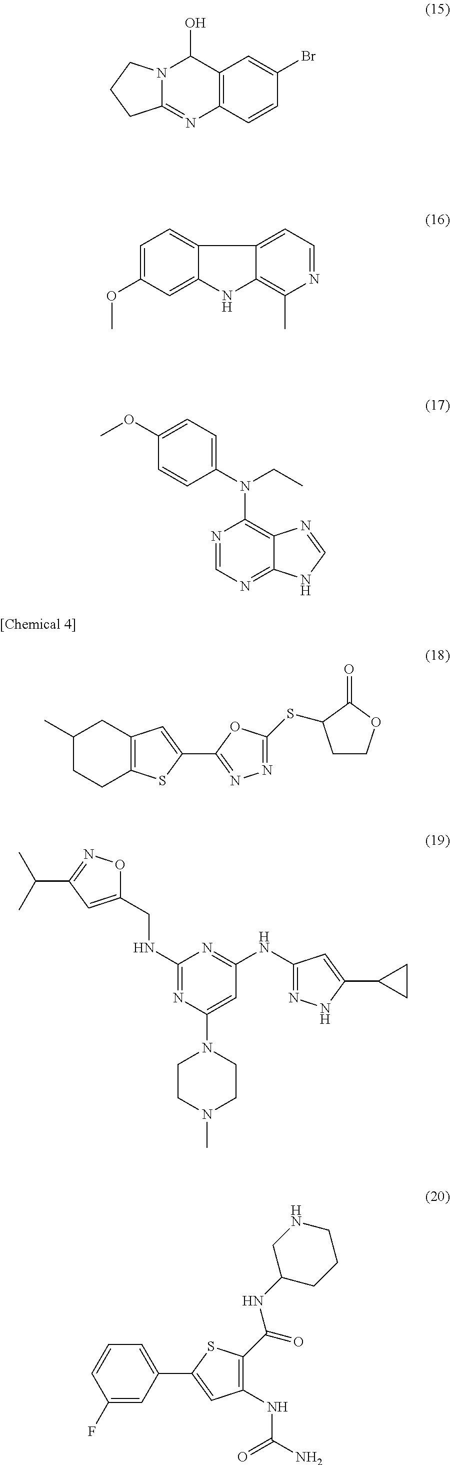 [SMILES: OC1c2cc(Br)ccc2N=C2CCCN21 ; COc1ccc2c(c1)[nH]c1c(C)nccc12 ; CCN(c1ccc(OC)cc1)c1ncnc2[nH]cnc12 ; CC1CCc2sc(-c3nnc(SC4CCOC4=O)o3)cc2C1 ; CC(C)c1cc(CNc2nc(Nc3cc(C4CC4)[nH]n3)cc(N3CCN(C)CC3)n2)on1 ; NC(=O)Nc1cc(-c2cccc(F)c2)sc1C(=O)NC1CCCNC1]

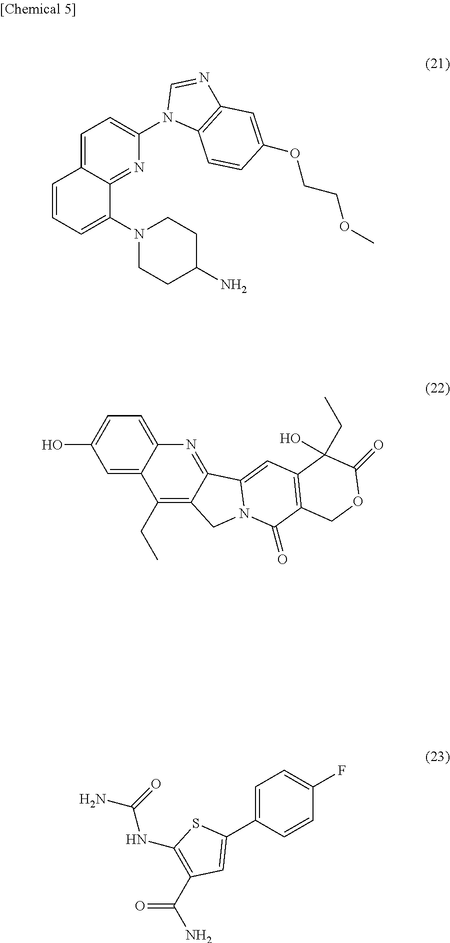 [SMILES: COCCOc1ccc2c(c1)ncn2-c1ccc2cccc(N3CCC(N)CC3)c2n1 ; CCc1c2c(nc3ccc(O)cc13)-c1cc3c(c(=O)n1C2)COC(=O)C3(O)CC ; NC(=O)Nc1sc(-c2ccc(F)cc2)cc1C(N)=O]

<2>A pharmaceutical composition for treating hypertriglyceridemia, containing:
the SREBP-1 inhibitor according to <1>; and
a pharmaceutically acceptable carrier.

### [Advantageous Effects of Invention]

According to the SREBP-1 inhibitor according to the above aspect, it is possible to provide an SREBP-1 inhibitor that specifically and selectively acts on SREBP-1. The pharmaceutical composition for treating hypertriglyceridemia according to the above aspect contains the SREBP-1 inhibitor, and thus it is expected to be effective in the treatment of hypertriglyceridemia.

### BRIEF DESCRIPTION OF DRAWINGS

FIG. 1 is a schematic block diagram of a vector in Example 1.

FIG. 2 is a graph showing the measurement results of luciferase activity in Example 1.

FIG. 3 is a schematic block diagram of a vector in Example 1.

FIG. 4 is a graph showing the measurement results of luciferase activity in Example 1.

FIG. 5 is an image showing the analysis results by westem blotting in Example 1.

FIG. 6A shows graphs showing the inhibition rates of SREBP-1 and SREBP-2 in the reporter assay in Example 1.

FIG. 6B shows graphs showing the inhibition rates of SREBP-1 and SREBP-2 in the reporter assay in Example 1.

FIG. 6C shows graphs showing the inhibition rates of SREBP-1 and SREBP-2 in the reporter assay in Example 1.

FIG. 6D shows graphs showing the inhibition rates of SREBP-1 and SREBP-2 in the reporter assay in Example 1.

FIG. 6E shows graphs showing the analysis results by western blotting in Example 1.

FIG. 6F shows graphs showing the analysis results by western blotting in Example 1.

FIG. 6G shows graphs showing the analysis results by western blotting in Example 1.

FIG. 6H shows graphs showing the analysis results by western blotting in Example 1.

FIG. 7A is a graph showing the triglyceride concentration in the culture solution in Example 2.

FIG. 7B is a graph showing the triglyceride concentration in the culture solution in Example 2.

FIG. 7C is a graph showing the triglyceride concentration in the culture solution in Example 2.

FIG. 7D is a graph showing the triglyceride concentration in the culture solution in Example 2.

FIG. 7E is a graph showing the triglyceride concentration in the culture solution in Example 2.

FIG. 7F is a graph showing the triglyceride concentration in the culture solution in Example 2.

FIG. 8 shows graphs showing the inhibition rates of SREBP-1 and SREBP-2 in the reporter assay in Example 3.

### DESCRIPTION OF EMBODIMENTS

### <<SREBP>>

Sterol regulatory element-binding protein (SREBP) is a transcription factor that belongs to the basic-helix-loop-helix-leucine zipper (bHLH-Zip) family. The vertebrate genome has homologous genes SREBP-1 and SREBP-2, both of which are expressed as a double-transmembrane precursor protein that is present on the membrane of the endoplasmic reticulum. SREBP and the SREBP cleavage-activating protein (SCAP) bind to each other through the C-terminals thereof and form a complex, and this SCAP functions as a cholesterol sensor. In a case where the cholesterol content in the membrane of the endoplasmic reticulum is less than 5%, SCAP binds to the coat protein II (CopII) protein through an amino acid sequence MELADL (SEQ ID NO: 1), whereby the SCAP/SREBP complex is transported to the Golgi apparatus. In the Golgi apparatus, in a case where SREBP is cleaved at two sites by proteolytic enzymes (SIP and S2P) in the periphery of the membrane-binding site, a portion on the N-terminal side (hereinafter, also referred to as nuclear type SREBP) is liberated, translocates into the nucleus, and binds to the sterol regulatory element (SRE), thereby acting as a transcription factor. SCAP is publicly known to be involved in the regulation of both SREBP-1/2 by cholesterol, whereas the SREBP-1-specific regulatory mechanism by EPA is largely unknown. According to our unpublished data, it is conceived that SCAP is not involved in the SREBP-1-specific regulation mechanism by EPA. An SREBP-1 inhibitor, such as EPA, in which the inhibitory action is not mediated by SCAP, has not been reported so far except for polyunsaturated fatty acids.

In SREBP-1, due to differences in promoters, there are two isoforms that are generated from exon 1a containing the start codon and exon 1c containing the same start codon, where the exon 1c is located at a position 14 kb downstream of the exon 1a. As a result, SREBP-1a and SREBP-1c have different N-terminal sides from each other, and SREBP-1c lacks 24 amino acid residues as compared with SREBP-1a. Due to having a short transcriptional activation region, SREBP-1c has weak transcription factor activity. In addition, the expression rates thereof vary from cell to cell. SREBP-1c and SREBP-2 are the major isoforms in cells of tissues such as the liver, and SREBP-1a is the major isoform in cultured cells or cells that proliferate actively. SREBP-1 and SREBP-2 each have a unique function although they have relatively high amino acid homology. Specifically, SREBP-1c controls the synthesis of fatty acids and triglycerides, SREBP-2 controls the synthesis of cholesterol, and SREBP-1a controls the synthesis of both fatty acids and cholesterol.

The inventors of the present invention revealed that EPA selectively inhibits the cleavage of SREBP-1 by acting on a portion located at the C-terminal side of SREBP-1. Based on this fact, as will be described later in examples, the inventors of the present invention constructed a proprietary reporter screening system in which SREBP-1 is used as a molecular target and Gal4 fused with the nuclear type SREBP-1 binds to a specific DNA sequence to exhibit luminescence, carried out a search by high-throughput screening using approximately 120,000 compounds in a library, and identified 23 kinds of compounds having an SREBP-1-specific inhibitory activity by excluding, from targets of interest by database search, compounds which have been reported to have a non-specific binding effect, thereby completing the present invention.

<<SREBP-1 inhibitor>

AN SREBP-1 inhibitor according to the present embodiment has an inhibitory activity against SREBP-1 and has no inhibitory activity against SREBP-2. The SREBP-1 inhibitor contains, as an active ingredient, any one or more compounds (hereinafter, each compound may be referred to as a “compound (1)” or the like) among compounds represented by Formulae (1) to (23).

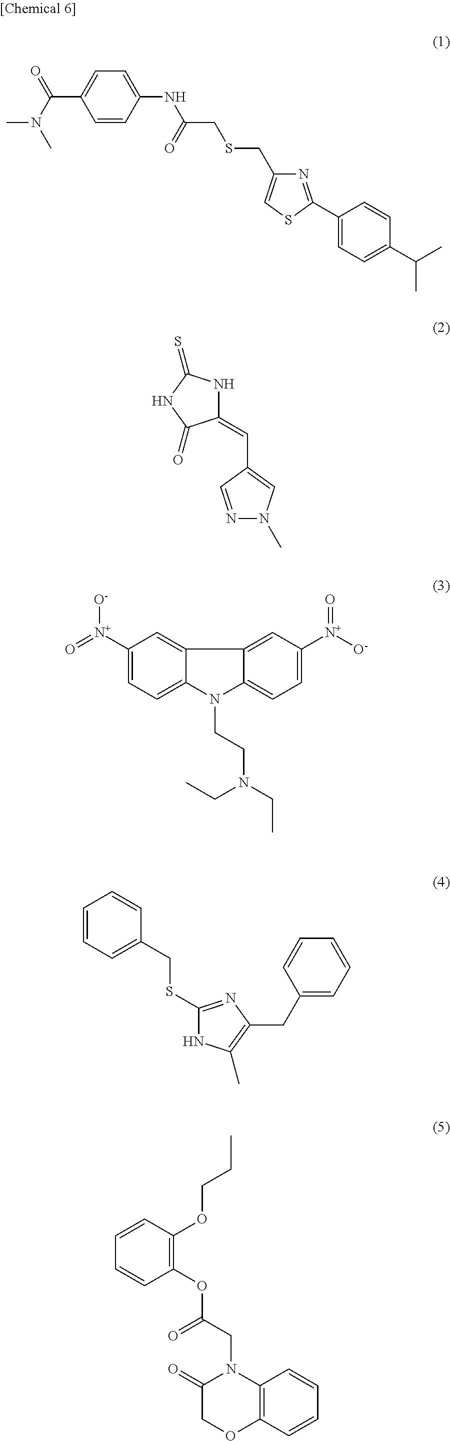 [SMILES: CC(C)c1ccc(-c2nc(CSCC(=O)Nc3ccc(C(=O)N(C)C)cc3)cs2)cc1 ; Cn1cc(/C=C2/NC(=S)NC2=O)cn1 ; CCN(CC)CCn1c2ccc([N+](=O)[O-])cc2c2cc([N+](=O)[O-])ccc21 ; Cc1[nH]c(SCc2ccccc2)nc1Cc1ccccc1 ; CCCOc1ccccc1OC(=O)CN1C(=O)COc2ccccc21]

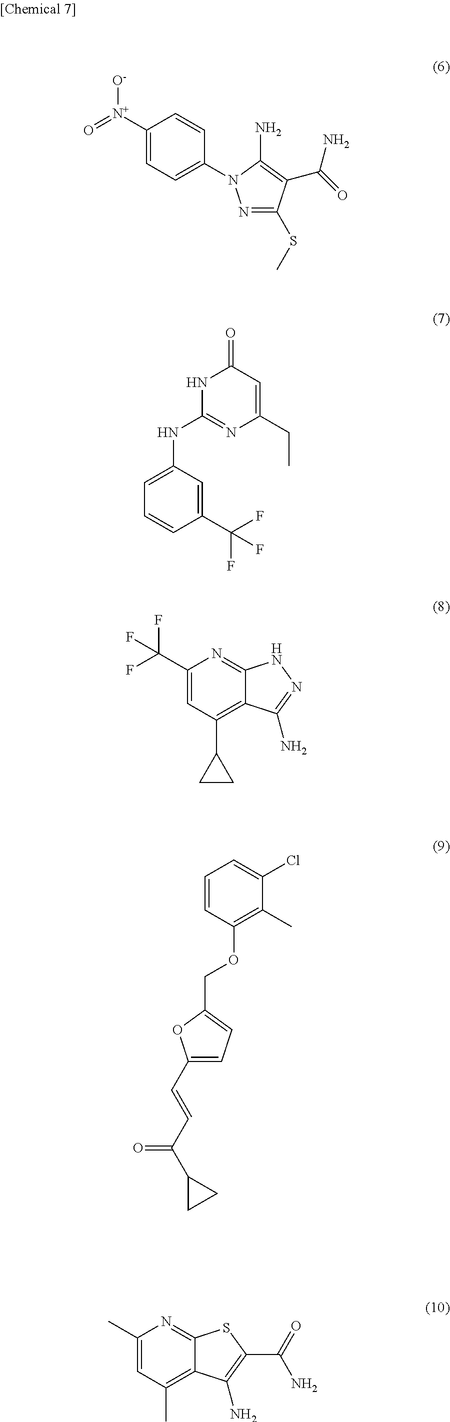

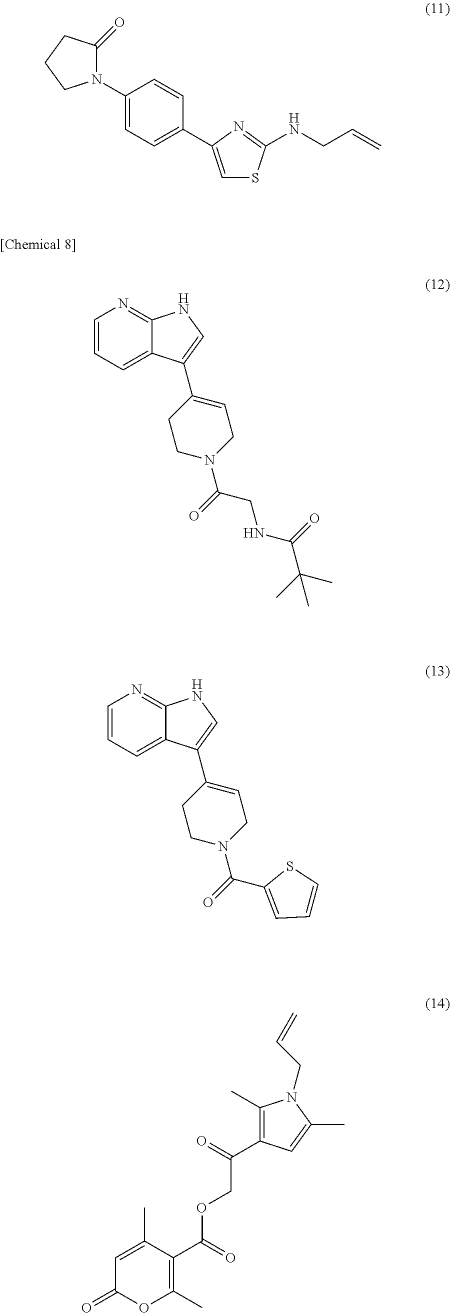 [SMILES: C=CCNc1nc(-c2ccc(N3CCCC3=O)cc2)cs1 ; CC(C)(C)C(=O)NCC(=O)N1CC=C(c2c[nH]c3ncccc23)CC1 ; O=C(c1cccs1)N1CC=C(c2c[nH]c3ncccc23)CC1 ; C=CCn1c(C)cc(C(=O)COC(=O)c2c(C)cc(=O)oc2C)c1C]

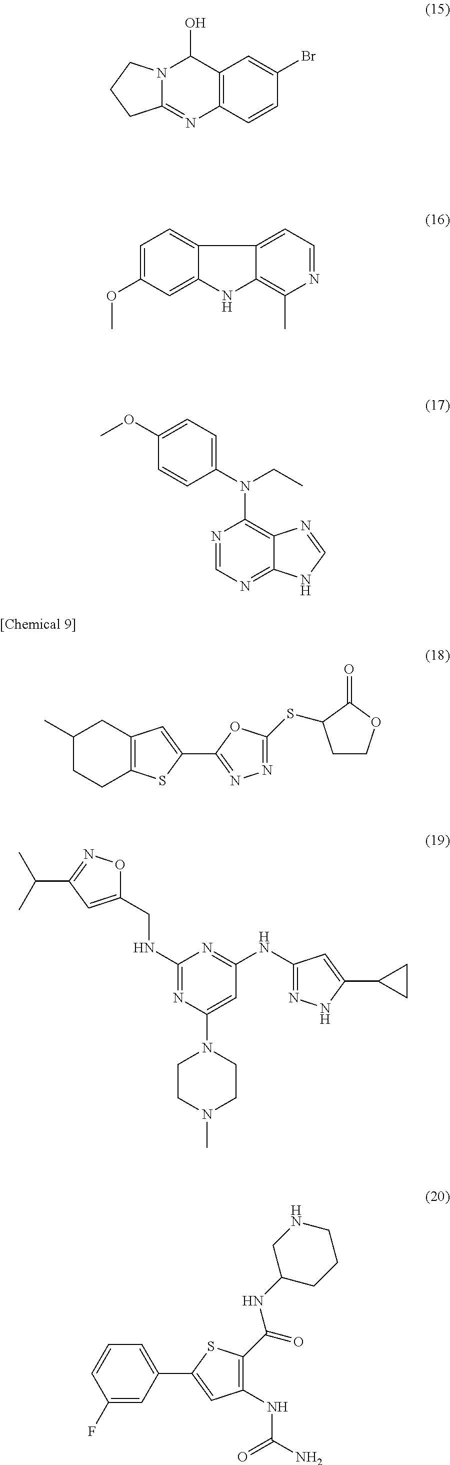 [SMILES: OC1c2cc(Br)ccc2N=C2CCCN21 ; COc1ccc2c(c1)[nH]c1c(C)nccc12 ; CCN(c1ccc(OC)cc1)c1ncnc2[nH]cnc12 ; CC1CCc2sc(-c3nnc(SC4CCOC4=O)o3)cc2C1 ; CC(C)c1cc(CNc2nc(Nc3cc(C4CC4)[nH]n3)cc(N3CCN(C)CC3)n2)on1 ; NC(=O)Nc1cc(-c2cccc(F)c2)sc1C(=O)NC1CCCNC1]

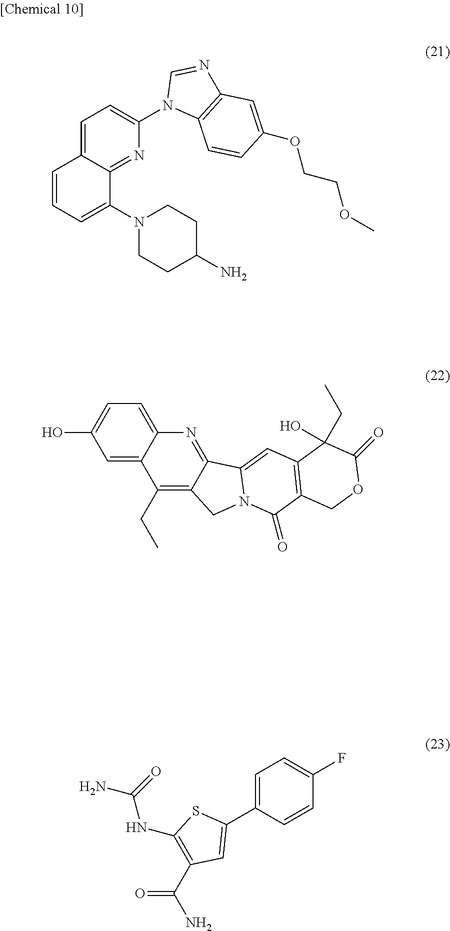 [SMILES: COCCOc1ccc2c(c1)ncn2-c1ccc2cccc(N3CCC(N)CC3)c2n1 ; CCc1c2c(nc3ccc(O)cc13)-c1cc3c(c(=O)n1C2)COC(=O)C3(O)CC ; NC(=O)Nc1sc(-c2ccc(F)cc2)cc1C(N)=O]

It is noted that in the present specification, the phrase “containing as an active ingredient” means containing a therapeutically effective amount of the compound. It is noted that in a case where two or more kinds of compounds are contained in combination, the total content thereof may be a therapeutically effective amount.

The compounds (1) to (23) are those that have specific inhibitory activity against SREBP-1 and no inhibitory activity against SREBP-2, as will be shown later in the examples. In addition, unlike fatty acids such as EPA, these compounds are expected to have degradation resistance in the body.

All of the compounds (1) to (23) are publicly known compounds. The details thereof are shown in Table 1 and Table 2 below.

TABLE 1

| Compound | IUPAC Name | CAS | PubChem URL |
|---|---|---|---|
| (1) | N,N-dimethyl-4-[[2-[[2-(4-propan-2-ylphenyl)- | | https://pubchem.ncbi.nlm.nih.gov/compound/38 [symbol] 30706 |
| | 1,3- [symbol] -4- | | |
| | yl] [symbol] mide | | |
| (2) | (5 [symbol] )-5-[(1-methylpyr [symbol] -4-yl) [symbol] -2- | | https://pubchem.ncbi.nlm.nih.gov/compound/0 [symbol] 273874 |
| | [symbol] -4-one | | |
| (3) | 2-(3,5- [symbol] -9-yl)-N,N- | | https://pubchem.ncbi.nlm.nih.gov/compound/ [symbol] 8258 [symbol] |
| | diethylethanamine | | |
| (4) | [symbol] -benzyl-2-benylsulfanyl- [symbol] -methyl- [symbol] - | | https://pubchem.ncbi.nlm.nih.gov/compound/72472 [symbol] |
| | i [symbol] ole | | |
| (5) | (2- [symbol] phenyl) 2-(3-oxo-1 [symbol] 4-benzo [symbol] -4- | | https://pubchem.ncbi.nlm.nih.gov/compound/243 [symbol] 4 [symbol] 36 |
| | yl) acetate | | |
| (6) | 5-amino-3-methylsulfanyl-1-(4- | | https://pubchem.ncbi.nlm.nih.gov/compound/ [symbol] 4638942 |
| | nitrophenyl)pyrazole-4-carboxamide | | |
| (7) | 4-ethyl-2-[3-(trifluoromethyl) [symbol] - [symbol] - | | https://pubchem.ncbi.nlm.nih.gov/compound/ [symbol] 35783 [symbol] 1 |
| | pyri [symbol] - [symbol] - [symbol] | | |
| (8) | 4-cyclopropyl- [symbol] -trifluoromethyl)-2H- | [symbol] 327 [symbol] -88-5 | https://pubchem.ncbi.nlm.nih.gov/compound/ [symbol] 024 [symbol] 41 |
| | [symbol] [3,4-b]pyridin-3-amine | | |
| (9) | ( [symbol] )-3-[ [symbol] -[(3-chloro-2- | | https://pubchem.ncbi.nlm.nih.gov/compound/ [symbol] 02298 [symbol] |
| | methyl [symbol] -2-yl [symbol] - | | |
| | [symbol] -2- [symbol] -1- [symbol] | | |
| (10) | 3-amino-4,6-dimethyl [symbol] [2 [symbol] 3- [symbol] din [symbol] -2- | 677 [symbol] 5-42-0 | https://pubchem.ncbi.nlm.nih.gov/compound/ [symbol] 13528 |
| | carboxamide | | |
| (11) | 1-[4-[2-(prop-2- [symbol] amino)-1 [symbol] 3- [symbol] -4- | | https://pubchem.ncbi.nlm.nih.gov/compound/94209 [symbol] 6 |
| | yl]phenyl] [symbol] din-2-one | | |
| (12) | 2,2-dimethyl-N-[2-oxo-2-[4-(1H-pyrro [symbol] [2,3- | | https://pubchem.ncbi.nlm.nih.gov/compound/ [symbol] 027364 |
| | [symbol] -3-yl)-3, [symbol] -dihydro-2H- [symbol] -1- | | |
| | yl]ethyl) [symbol] mid [symbol] | | |

[symbol]

indicates data missing or illegible when filed

TABLE 2

| Compound | IUPAC Name | CAS | PubChem URL |
|---|---|---|---|
| (13) | [4-(1H-pyrrolo[2,3-b]pyridin-3-yl)-3,6- | | https://pubchem.ncbi.nlm.nih.gov/compound/40138089 |
| | dihydro-2H-pyridin-1-yl]-thiophen-2- | | |
| | yl [symbol] | | |
| (14) | [2-(2,5-dimethyl-1-prop-2-enylpy [symbol] -3-yl)-2- | | https://pubchem.ncbi.nlm.nih.gov/compound/245338 [symbol] |
| | oxoethyl] 2,4-dimethyl- [symbol] - [symbol] -3- | | |
| | carboxylate | | |
| (15) | [symbol] -bromo-1,2,3, [symbol] -tetrahydropy [symbol] [2,1- | | https://pubchem.ncbi.nlm.nih.gov/compound/371 [symbol] 338 |
| | b] [symbol] olin-9-ol | | |
| (16) | [symbol] - [symbol] thoxy-1-methyl- [symbol] H- [symbol] [3,4-b] indole | 442-51-3 | https://pubchem.ncbi.nlm.nih.gov/compound/ [symbol] 2 [symbol] 0 [symbol] 3 |
| (17) | N-ethyl-N-(4-methoxyphenyl)-7H- [symbol] -5-amino | | https://pubchem.ncbi.nlm.nih.gov/compound/756730 |
| (18) | 3-[[5-(5-methyl-4,5,6,7-tetrahydro- [symbol] - | | https://pubchem.ncbi.nlm.nih.gov/compound/183238 [symbol] |
| | [symbol] enz [symbol] -2-yl)-1,3,4- [symbol] zol-2- | | |
| | yl) [symbol] fanyl] [symbol] -2- [symbol] | | |
| (19) | 4-N-(5-cyclopropyl- [symbol] -py [symbol] ol-3-yl)- [symbol] -(4- | [symbol] 8280-07-4 | https://pubchem.ncbi.nlm.nih.gov/compound/597 [symbol] 74 |
| | methylpi [symbol] in-1-yl)-2-N-[(3-pr [symbol] -2-yl- | | |
| | 1,2- [symbol] -5-yl) [symbol] -2,4-diamine | | |
| (20) | 3-( [symbol] yl [symbol] in [symbol] )- [symbol] -(3- [symbol] phenyl)-N- | | https://pubchem.ncbi.nlm.nih.gov/compound/1 [symbol] 041 [symbol] 0 |
| | piperidin-3- [symbol] -2-carboxamide | | |
| (21) | 1-(2-(5- [symbol] 2-Methoxy [symbol] ylbe [symbol] imi [symbol] azol-1- | 343787-2 [symbol] -1 | https://pubchem.ncbi.nlm.nih.gov/compound/1015 [symbol] 0 |
| | yl) [symbol] in [symbol] -8-yl)piperidin-4-ylamine | | |
| (22) | ( [symbol] )- [symbol] -di [symbol] thyl- [symbol] -dihydroxy- [symbol] - [symbol] - | 86 [symbol] 3 [symbol] -52-3 | https://pubchem.ncbi.nlm.nih.gov/compound/1 [symbol] 4842 |
| | 3,13- | | |
| | di [symbol] 02 [symbol] 04 [symbol] | | |
| | [symbol] - [symbol] (21),2,4(9) [symbol] 5,7,10,15(20)- [symbol] - | | |
| | 14,18-dion [symbol] | | |
| (23) | 2-(car [symbol] ylamino)-5-(4- | [symbol] 07475-17-4 | https://pubchem.ncbi.nlm.nih.gov/compound/ [symbol] 0378 [symbol] |
| | fluorophenyl) [symbol] -3-carboxamide | | |

[symbol]

indicates data missing or illegible when filed

In addition, each of the compounds (1) to (23) may be in the form of a salt or cocrystal. Examples of the salt of each compound include a salt of an inorganic acid and a salt of an organic acid, where the salt is a salt suitable for the separation or crystallization of each compound. Specific examples of the salt of each compound include an organic amine salt, an alkali metal salt, an ammonium salt, and another pharmaceutically acceptable salt.

Examples of other pharmaceutically acceptable salts of each compound include a hydrochloride, a bromate, a sulfate, a hydrogen sulfate, a dihydrogen phosphate, a methanesulfonate, a methyl sulfate, a maleate, a fumarate, a 2-naphthalenesulfonate, a benzenesulfonate, a glycolate, a gluconate, a citrate, an isethionate, and a p-toluenesulfonate.

Examples of the additive for cocrystals of each compound include oxalic acid, trimethyl glycine, and other pharmaceutically acceptable compounds.

Each of the compounds (1) to (23) to be used may be a commercially available compound or may be synthesized according to a publicly known synthesis method.

<<Pharmaceutical composition for treating hypertriglyceridemia>>

The pharmaceutical composition for treating hypertriglyceridemia according to the present embodiment (which may be simply referred to hereinafter as the pharmaceutical composition according to the present embodiment) contains the SREBP-1 inhibitor and a pharmaceutically acceptable carrier.

The pharmaceutical composition according to the present embodiment is expected to be effective in the treatment of hypertriglyceridemia.

Since the administration of the SREBP-1 inhibitor can reduce the blood triglyceride level, the disease to which the pharmaceutical composition according to the present embodiment is applied is primarily hypertriglyceridemia; however, the pharmaceutical composition according to the present embodiment can also be suitably applied to other diseases associated with the elevation of blood triglyceride levels. Specifically, it can also be suitably used as a pharmaceutical composition for treating fatty liver and arteriosclerosis.

<Pharmaceutically acceptable carrier>

As the pharmaceutically acceptable carrier, a carrier that is used for the pharmaceutical preparation of a general pharmaceutical composition can be used without particular limitation. More specific examples thereof include binding agents such as gelatin, corn starch, tragacanth gum, and gum arabic: excipients such as starch and crystalline cellulose: swelling agents such as alginic acid: solvents for an injection agent, such as water, ethanol, and glycerin; and pressure-sensitive adhesives such as a rubber-based pressure-sensitive adhesive and a silicone-based pressure-sensitive adhesive.

The pharmaceutical composition according to the present embodiment may contain an additive. Examples of the additive include lubricants such as calcium stearate and magnesium stearate: sweetening agents such as sucrose, lactose, saccharin, and maltitol; flavoring agents such as peppermint and Gaultheria adenothrix oil; stabilizers such as benzyl alcohol and phenol: buffering agents such as a phosphoric acid salt and sodium acetate: dissolution assisting agents such as benzyl benzoate and benzyl alcohol: antioxidants; and preservatives.

The pharmaceutical composition according to the present embodiment can be formed into a pharmaceutical preparation by suitably combining the SREBP-1 inhibitor, the pharmaceutically acceptable carrier, and an additive and then mixing them in a unit dosage form that is required for generally accepted pharmaceutical practice.

The pharmaceutical composition according to the present embodiment may be used in combination with a publicly known therapeutic drug for hypertriglyceridemia other than the SREBP-1 inhibitor. The SREBP-1 inhibitor and other drugs may be formed into the same pharmaceutical preparation or may be formed into separate pharmaceutical preparations. In addition, each pharmaceutical preparation may be administered through the same administration route or may be administered through administration routes different from each other. Further, each pharmaceutical preparation may be administered simultaneously, administered sequentially, or administered separately after an interval of a predetermined number of hours or a predetermined period. In one embodiment, the SREBP-1 inhibitor and other drugs may be provided to be contained in a kit.

<Method of administration>

The target to be administered is not limited; however, examples thereof include a human, a monkey, a dog, a cow, a horse, a sheep, a pig, a rabbit, a mouse, a rat, a guinea pig, a hamster, and cells thereof. Among them, a mammal or a mammalian cell is preferable, and a human or a human cell is particularly preferable.

The dose of the pharmaceutical composition according to the present embodiment varies depending on the kind of compound, the symptom of the administration subject, the administration site, the method of administration, and the like. An appropriate dose may be appropriately selected by those skilled in the art.

The administration of the pharmaceutical composition according to the present embodiment may be a single administration or may be a plurality of times of administration. In a case of a plurality of times of administration, the administration can be carried out at a frequency, for example, every period of 2 hours or more and 12 hours or less, every day, or every two days, every five days, every one week, every 1.5 weeks, every several weeks, every one month, or every several months.

### OTHER EMBODIMENTS

In one embodiment, the present invention provides a medical treatment method for hypertriglyceridemia, including administering an effective amount of an SREBP-1 inhibitor to a patient or affected animal in need of treatment. Here, examples of the SREBP-1 inhibitor include the same ones as those described above.

In one embodiment, the present invention provides an SREBP-1 inhibitor for the treatment of hypertriglyceridemia. Here, examples of the SREBP-1 inhibitor include the same ones as those described above.

In one embodiment, the present invention provides use of any one or more compounds among the compounds (1) to (23) for producing an SREBP-1 inhibitor. Here, examples of the SREBP-1 inhibitor include the same ones as those described above.

### EXAMPLES

The present invention will be described with reference to Examples; however, the present invention is not limited to Examples below.

### Example 1

The inventors of the present invention aimed to develop a novel therapeutic drug that is more excellent than EPA in the specificity of inhibitory activity against SREBP-1, and first, constructed a reporter screening system that targets SREBP-1 as a molecular target and exhibits luminescence in a case where the nuclear type SREBP-1 binds to SRE.

1. Selection of SREBP-1 inhibiting compound
(1) Vector construction

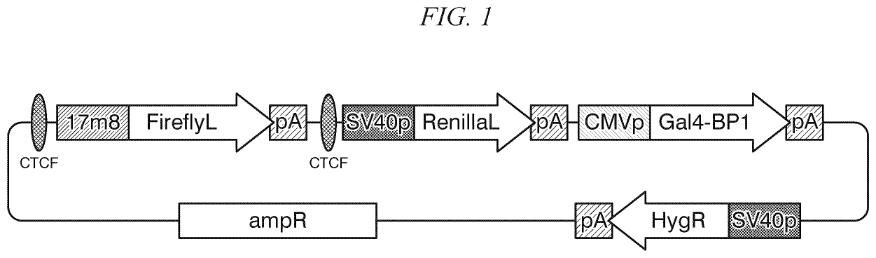

A pcDNA3.1 (+)-Hyg vector (manufactured by Thermo Fisher Scientific, Inc. under the brand of Invitrogen) was inserted with DNA fragments of a firefly luciferase reporter cloned from a pGL3-Basic vector (manufactured by Promega Corporation), a pSV40-Renilla luciferase reporter cloned from a pRL-SV40 vector (manufactured by Promega Corporation), and a human SREBP-1c cloned from a pTK-HSV-hSREBP-1c, whereby a vector having a structure shown in FIG. 1 was constructed. The base sequence of the vector is set forth in SEQ ID NO: 2. In FIG. 1, Gal4-BP1 is a human SREBP-1c that is obtained by fusing sequences of the Gal4-DNA binding domain and the minimal VP16-AD to the N-terminal side. 17 m8 is a DNA sequence to which the Gal4-DNA binding domain binds. CTCF is a DNA sequence that acts as an insulator, which blocks the action of transcription factors.

(2) Establishment of stable expression strain of HepG2

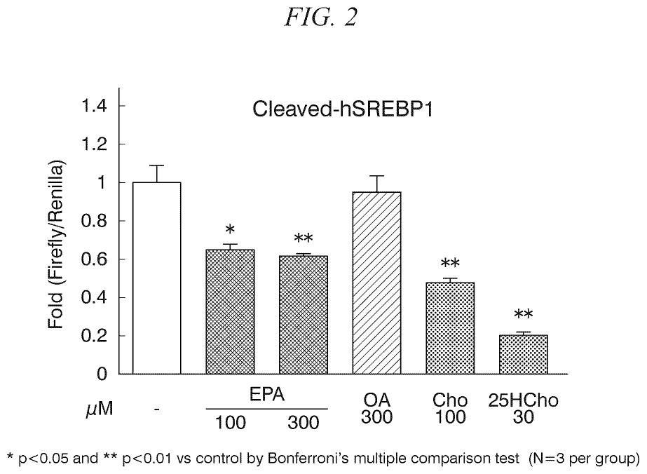

HepG2 cells, which are a cell line derived from human liver cancer, were cultured in advance to a confluency of 10% confluent in Dulbecco's Modified Eagle's Medium (DMEM) (containing 25 mM glucose, 100 units/mL penicillin, 100 g/mL streptomycin sulfate, and 10 v/v % fetal bovine serum (FBS)) and then transfected with the vector obtained in (1) described above, by using a Lipofectamine 3000 transfection reagent (manufactured by Thermo Fisher Scientific, Inc. under the brand of Invitrogen), 5 whereby the vector was inserted into the genome. Selection was carried out with hygromycin to establish a stable expression strain of HepG2 which stably expresses the firefly luciferase. To confirm that the stable expression strain of HepG2 could be established, EPA (concentration in culture medium: 100 μM or 300 μM), oleic acid (OA; concentration in culture medium: 300 μM), cholesterol (Cho; concentration in culture medium: 100 μM), or 25-hydroxycholesterol (25HCho; concentration in culture medium; 30 μM) was added and incubated for 24 hours. Next, a Reporter lysis buffer (Promega Corporation) was added to lyse the cells, and luciferase activity was measured with a luminometer. The results are shown in FIG. 2.

As shown in FIG. 2, luciferase luminescence was observed in a compound-free group (a group in which dimethyl sulfoxide (DMSO) was added as a control) and an OA addition group, whereas luciferase activity was reduced in an EPA addition group, a Cho addition group, and a 25HCho addition group. This made it possible to confirm that the established cell line can be applied to a screening of compounds having an inhibitory action on SREBP-1.

(3) High-throughput screening

Next, each candidate compound (concentration in culture medium: 2 or 5 μM) was added to the cell line (2×10 4 cells/well) seeded in a plurality of 384-well plates, which had been constructed in (2) described above, so that one kind of each compound was added to one well, and incubation was carried out for 24 hours. As the candidate compound, 11.9307 kinds of low molecular weight compounds registered in the full library of Drug Discovery Initiative, the University of Tokyo, were used. Next, Steady-Glo (a mixture of luciferin and a Lysis buffer) was added thereto, and then luciferase activity was measured with a luminometer. As a result, 450 kinds of compounds were selected as compounds exhibiting inhibitory action on SREBP-1.

2. Exclusion of SREBP-2 inhibiting compound
(1) Vector construction

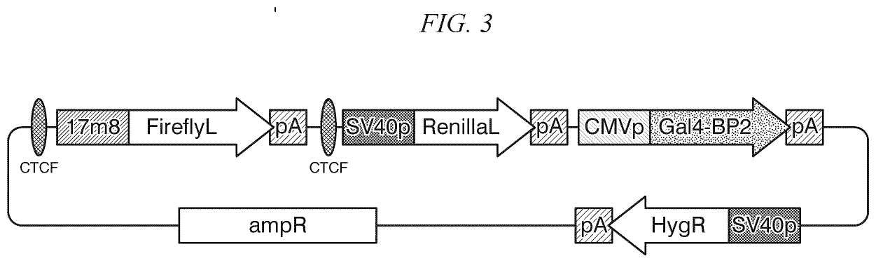

In order to exclude compounds such as cholesterol and 25-hydroxycholesterol, which have an inhibitory action on both SREBP-1 and SREBP-2, first, a vector having the structure shown in FIG. 3 was constructed using the same method as in “1.” described above, except that a DNA fragment of human SREBP-2 cloned from pTK-HSVhSREBP-2 was used instead of the DNA fragment of human SREBP-1c. The base sequence of the vector is set forth in SEQ ID NO: 3. In FIG. 3, Gal4-BP2 is a human SREBP-2 that is obtained by fusing sequences of the Gal4-DNA binding domain and the minimal VP16-AD to the N-terminal side.

(2) Establishment of stable expression strain of HepG2

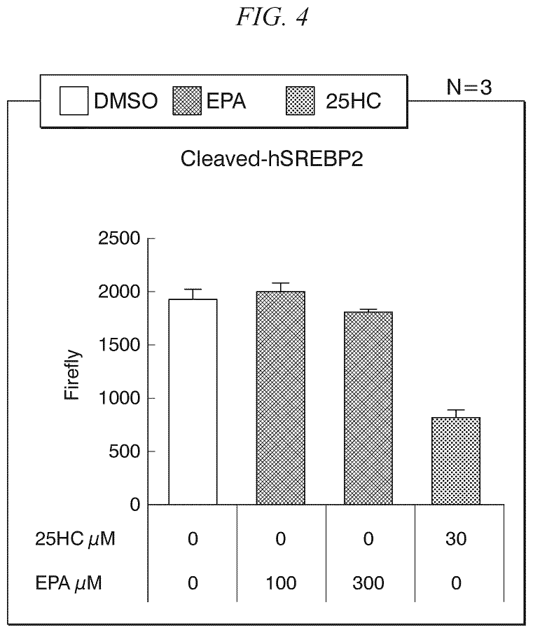

a stable expression strain of HepG2, which stably expressed firefly luciferase, was established using the same method as in “1. (2)” described above, except that the vector obtained in “2. (1)” described above was used. To confirm that the stable expression strain of HepG2 could be established, EPA (concentration in culture medium; 100 μM or 300 μM) or 25-hydroxycholesterol (25HCho, concentration in culture medium: 30 μM), and incubation was carried out for 24 hours. Next, a Reporter lysis buffer (Promega Corporation) was added to lyse the cells, and luciferase activity was measured with a luminometer. The results are shown in FIG. 4.

As shown in FIG. 4, luciferase luminescence was observed in a compound-free group (a group in which DMSO was added as a control) and an EPA addition group, whereas luciferase activity was reduced in a 25HCho addition group. This made it possible to confirm that the established cell line can be applied to a screening for excluding compounds having an inhibitory action on SREBP-2.

### (3) High-Throughput Screening

Next, a high-throughput screening was carried out using the same method as in “1. (3)” described above, except that the cell line established in “2. (2)” described above was used and that each of the 450 kinds of candidate compounds selected in “1. (3)” was added. As a result, compounds having an inhibitory action on SREBP-2 were excluded, and 77 kinds of compounds were selected as compounds having a selective inhibitory action on SREBP-1.

### 3. Selection by Western Blotting

Selection by Western blotting was carried out in order to confirm the inhibitory action on SREBP-1 from the expression level of the SREBP-1 protein.

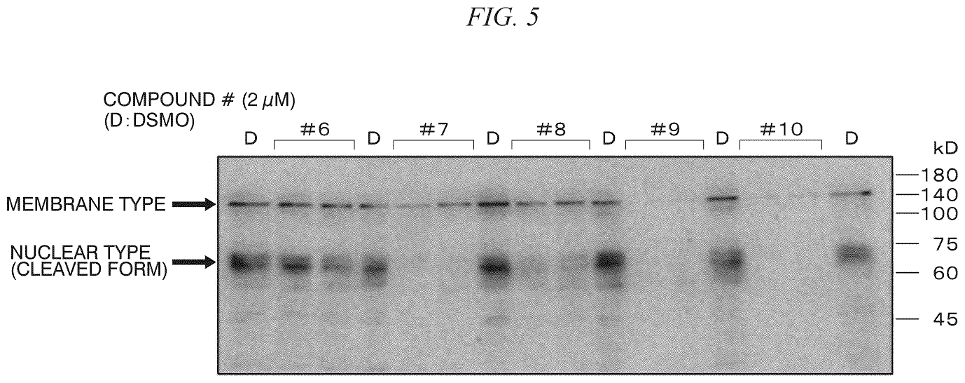

First, H4IIEC3 cells (1×10⁵ cells/well) seeded in a 48-well plate, which are a cell line of rat hepatocytes, were cultured for 1 day. Next, culture medium exchange was carried out so that a high glucose DMEM (serum-free) containing each candidate compound (concentration in culture medium: 2 μM) or DMSO as a control was 200 μL/well, and then the cells were cultured for 24 hours. Next, the cells were recovered, and the cells were lysed in 50 μL of a Cell Lysis buffer (50 mM Tris-HCl, pH: 7.5, 137 mM NaCl, 1 mM EDTA, Triton X: 1%) and allowed to stand on ice for 30 minutes. Next, centrifugation was carried out at 15,000 rpm for 10 minutes at 4° C. to obtain 35 μL of a cell lysate as the supernatant. Next, SDS-PAGE (10 w/w % acrylamide gel) was carried out using 15 μL of cell lysate, and the transfer was carried out to a polyvinylidene fluoride (PVDF) membrane according to a semi-dry method. Next, the membrane after the transfer was blocked with TBS-tween containing 5 w/v % skim milk and allowed to react overnight at 4° C. with a primary antibody (anti-SREBP-1 antibody: sc-135512A4 mouse, manufactured by Santa Cruz Biotechnology, Inc., diluted 1/100 with TBS-tween containing 1 w/v % skim milk). Next, it was washed 3 times with TBS-tween and allowed to react for 1 hour at room temperature (about 25° C.) with a secondary antibody (anti-mouse IgG-HRP conjugate antibody: CST7076, diluted 1/2,000 with TBS-tween containing 5 w/v % skim milk). Next, washing was carried out three times with TBS-tween, color development was carried out with a chemiluminescence reagent (PerkinElmer, Inc., NEL104001EA), and then measurement was carried out with a CCD imager (LAS4000). A part of the results for the candidate compounds according to Western blotting are shown in FIG. 5.

Among the 77 kinds of candidate compounds, 39 kinds of compounds were selected as compounds by which a decrease in the expression level of SREBP-1 was confirmed by Western blotting analysis.

4. Exclusion of Compounds for which Nonspecific Action is Reported

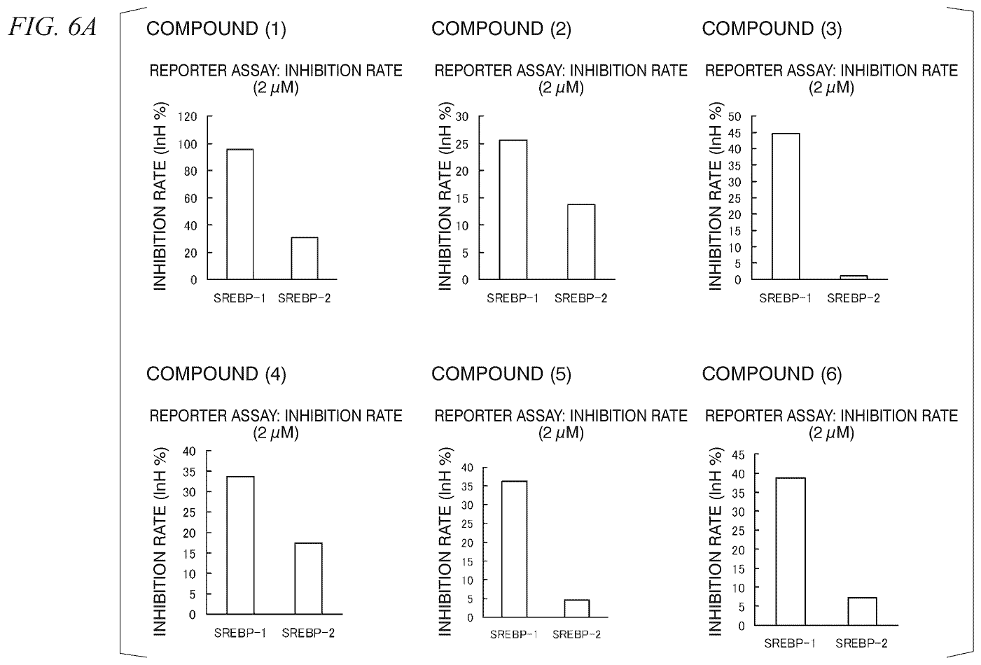

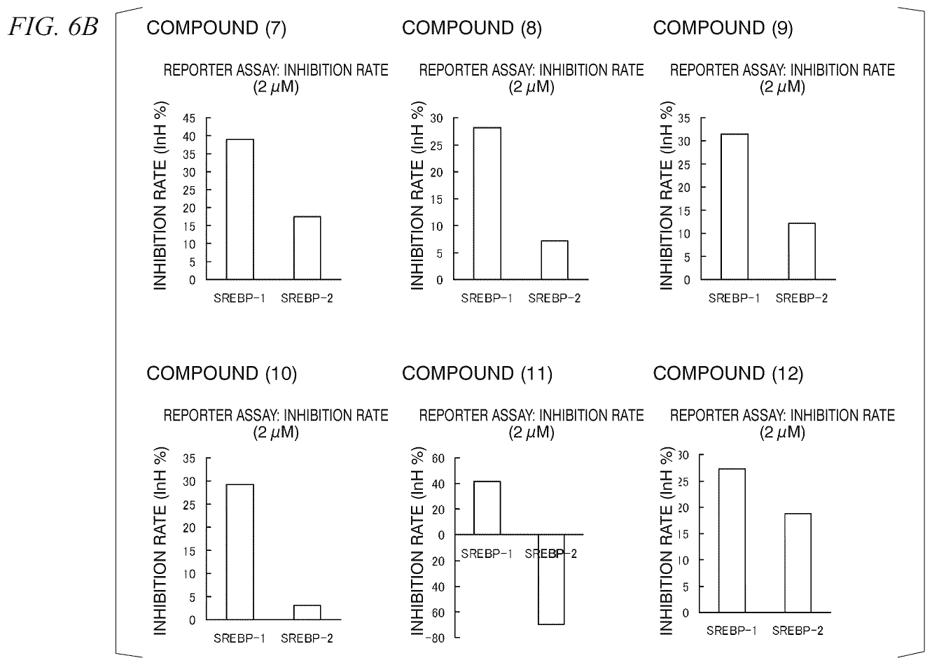

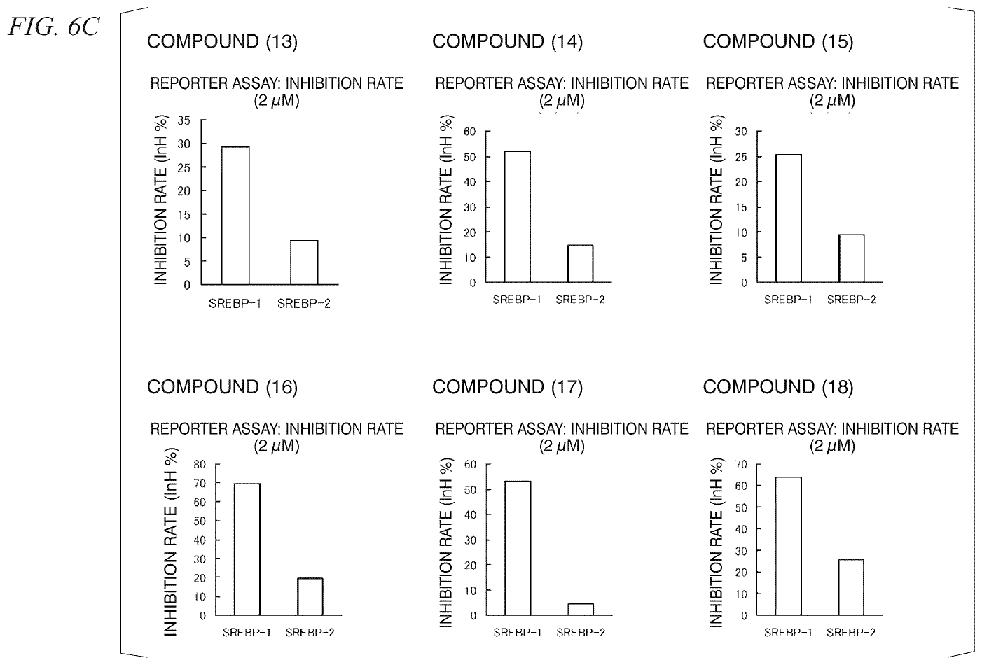

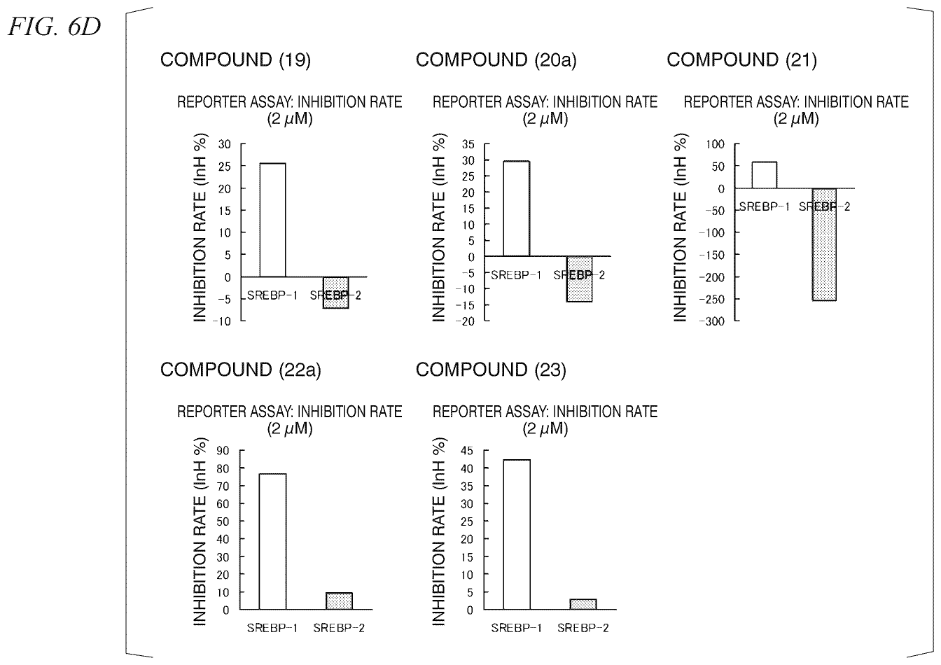

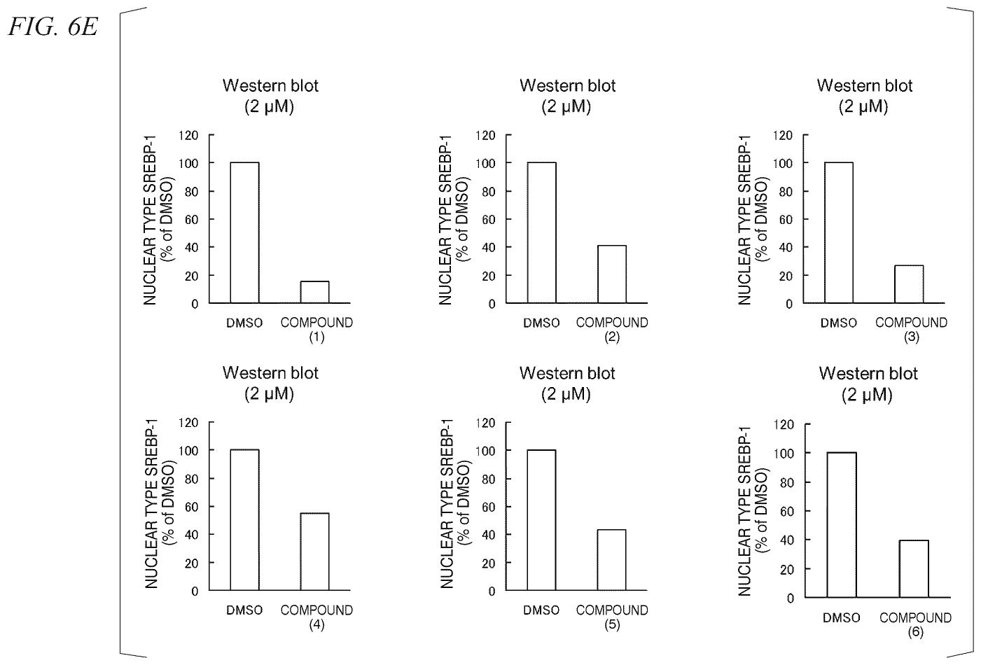

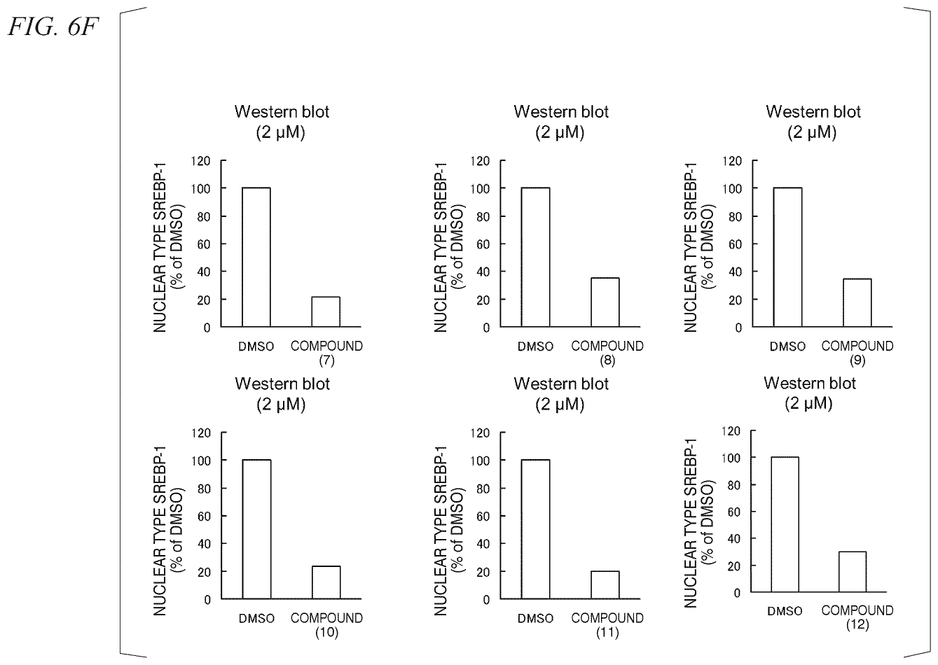

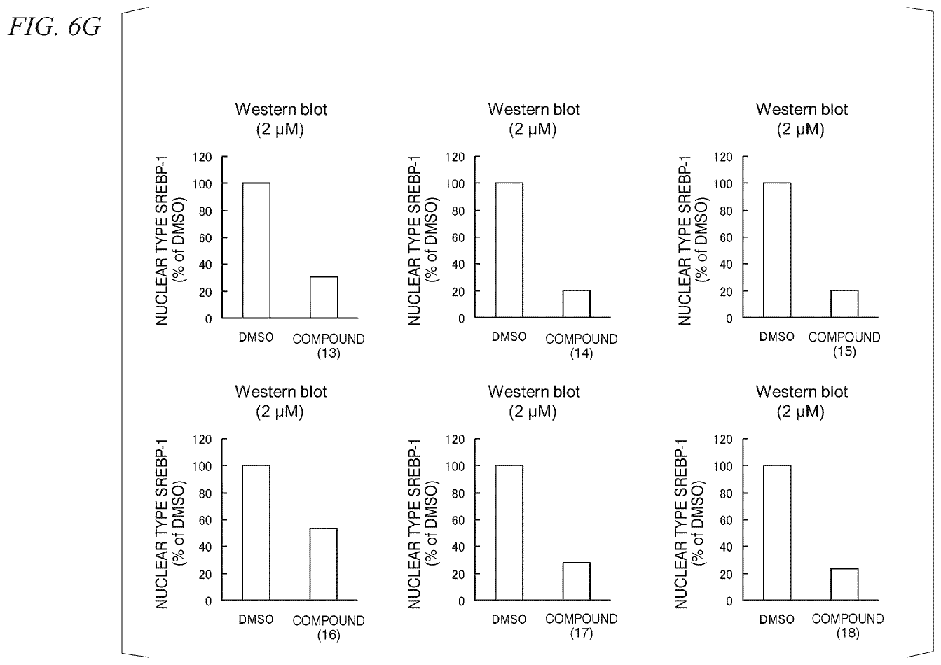

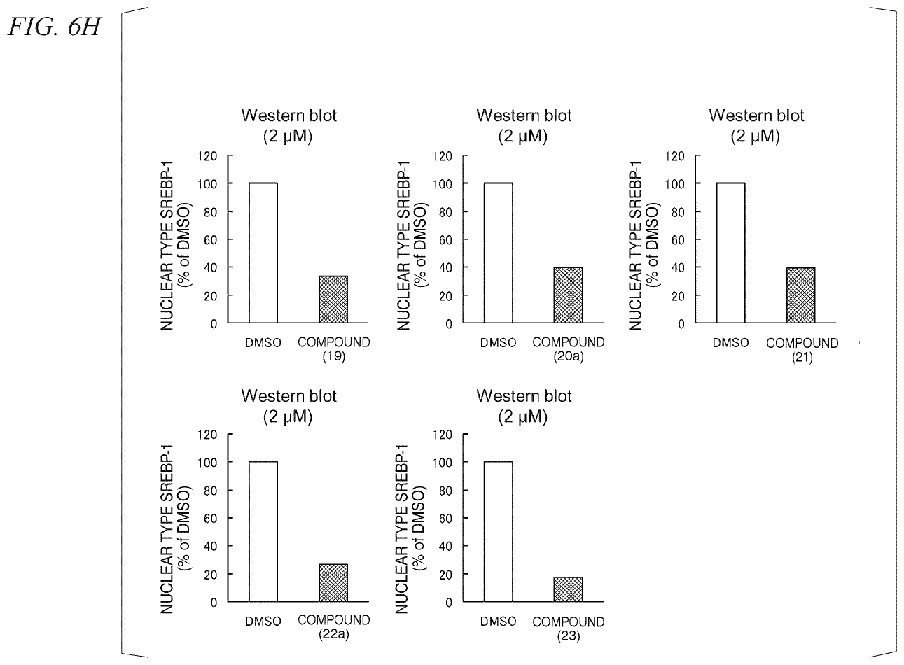

Among the 39 kinds of compounds, 23 kinds of compounds were selected by searching the PAINS database (https://www.rdkit.org/docs/source/rdkit. Chem.rdfiltercatalog.html) and excluding, from targets of interest, compounds for which non-specific action had been reported. Details of the 23 kinds of compounds are given below. In addition, FIG. 6A to FIG. 6D each shows a graph showing the SREBP-1 inhibition rate and the SREBP-2 inhibition rate in the reporter assay for each compound, and FIG. 6E to FIG. 6H each shows a graph of the results of the Western blotting analysis using anti-SREBP-1 antibody. It is noted that the SREBP-1 inhibition rate and the SREBP-2 inhibition rate in the reporter assay were calculated using the following method. That is, the inhibition rate (InH %) was calculated according to the following expression from the values of Sample (an average value of measured values of luciferase activity in wells in which each compound was added to the cells), Control (an average value of measured values of luciferase activity in wells in which only DMSO used as a solvent was added to the cells), and Background (an average value of measured values of luciferase activity in wells in which only DMSO was added without seeding the cells).

$${}^{″} InH {( \% )}^{″} = [ 1 - \{ ( Sample ) - ( Background ) \} / \{ ( Control ) - ( Background ) \} ] \times 100$$

These results indicate that all of the 23 kinds of compounds have inhibitory activity against SREBP-1, which is specific and selective.

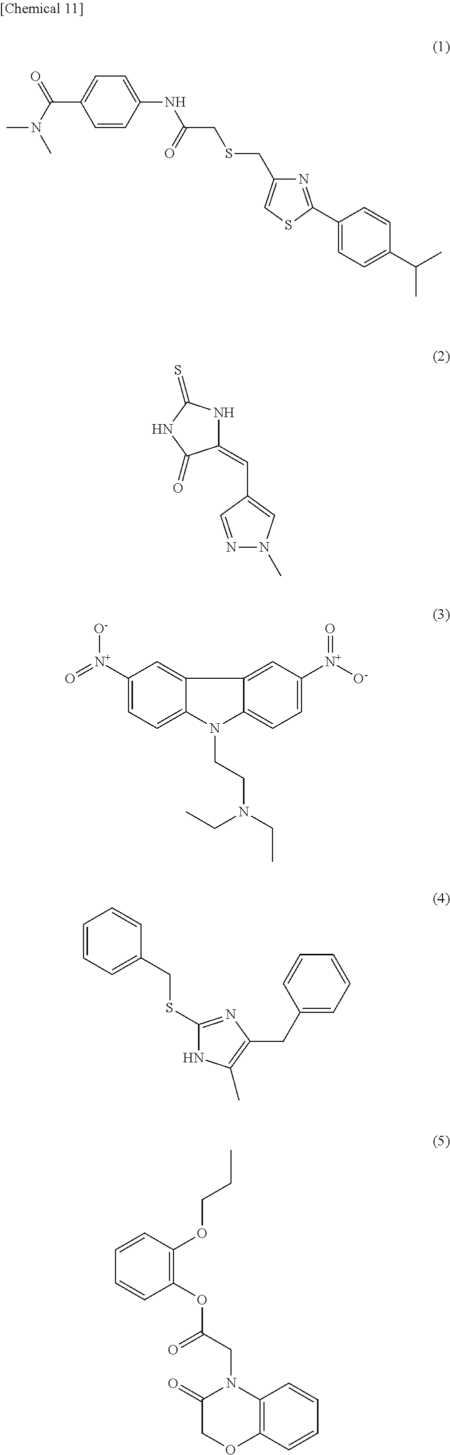 [SMILES: CC(C)c1ccc(-c2nc(CSCC(=O)Nc3ccc(C(=O)N(C)C)cc3)cs2)cc1 ; Cn1cc(/C=C2/NC(=S)NC2=O)cn1 ; CCN(CC)CCn1c2ccc([N+](=O)[O-])cc2c2cc([N+](=O)[O-])ccc21 ; Cc1[nH]c(SCc2ccccc2)nc1Cc1ccccc1 ; CCCOc1ccccc1OC(=O)CN1C(=O)COc2ccccc21]

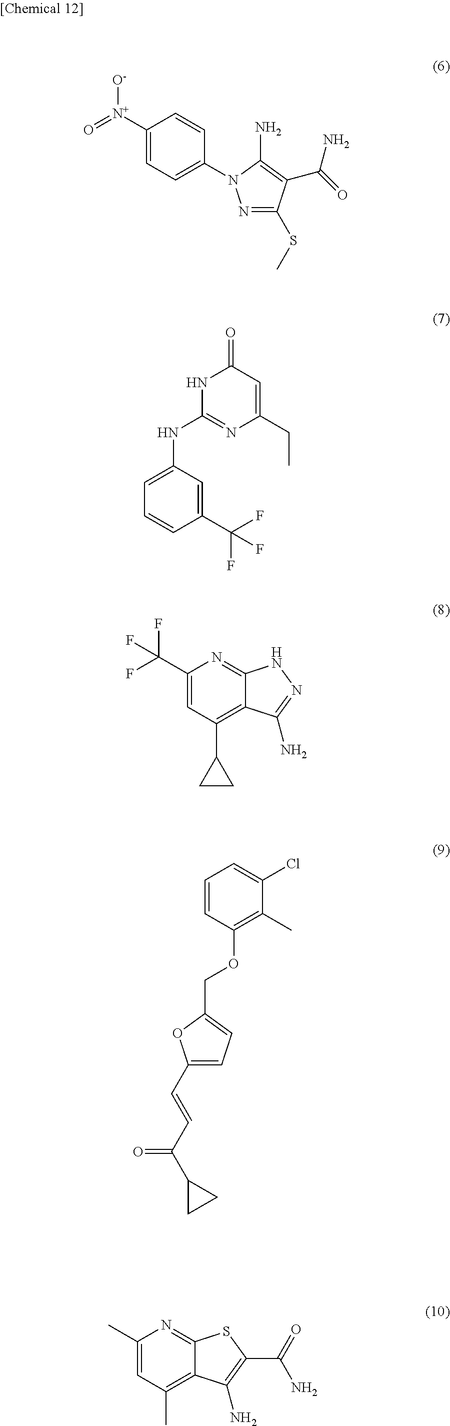

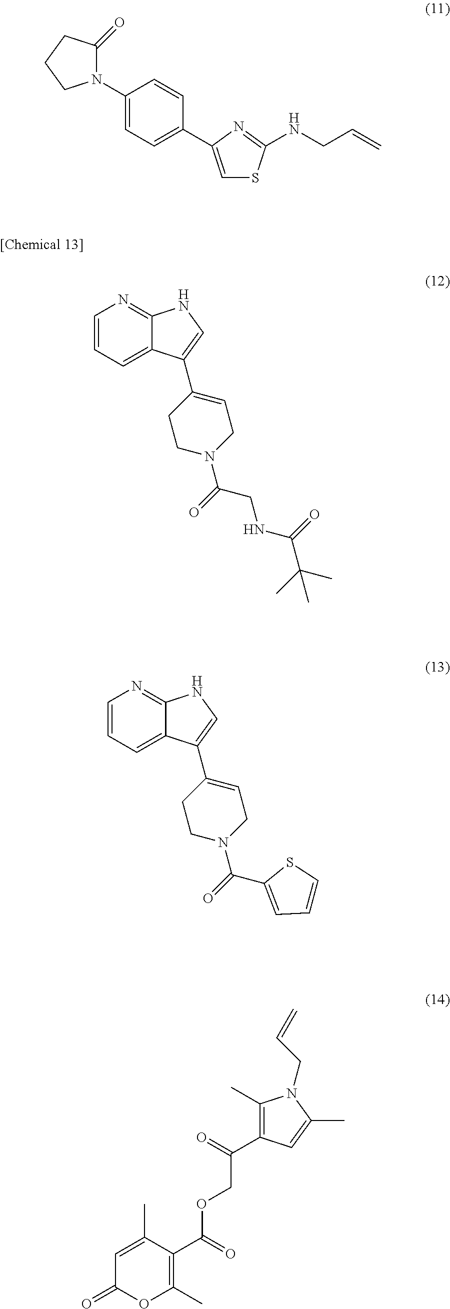 [SMILES: C=CCNc1nc(-c2ccc(N3CCCC3=O)cc2)cs1 ; CC(C)(C)C(=O)NCC(=O)N1CC=C(c2c[nH]c3ncccc23)CC1 ; O=C(c1cccs1)N1CC=C(c2c[nH]c3ncccc23)CC1 ; C=CCn1c(C)cc(C(=O)COC(=O)c2c(C)cc(=O)oc2C)c1C]

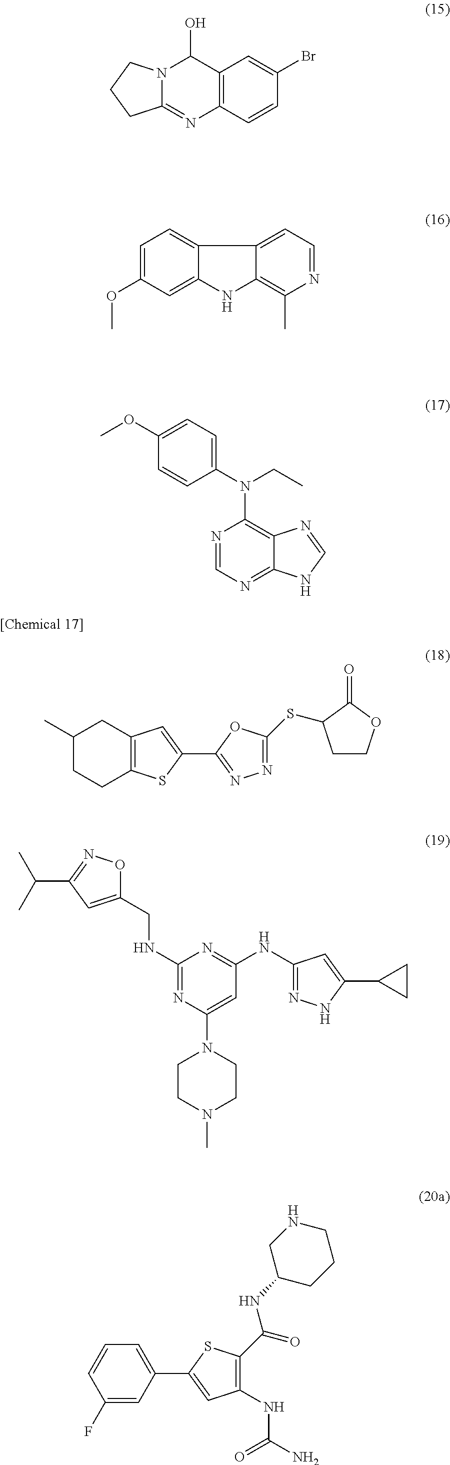 [SMILES: OC1c2cc(Br)ccc2N=C2CCCN21 ; COc1ccc2c(c1)[nH]c1c(C)nccc12 ; CCN(c1ccc(OC)cc1)c1ncnc2[nH]cnc12 ; CC1CCc2sc(-c3nnc(SC4CCOC4=O)o3)cc2C1 ; CC(C)c1cc(CNc2nc(Nc3cc(C4CC4)[nH]n3)cc(N3CCN(C)CC3)n2)on1 ; NC(=O)Nc1cc(-c2cccc(F)c2)sc1C(=O)N[C@H]1CCCNC1]

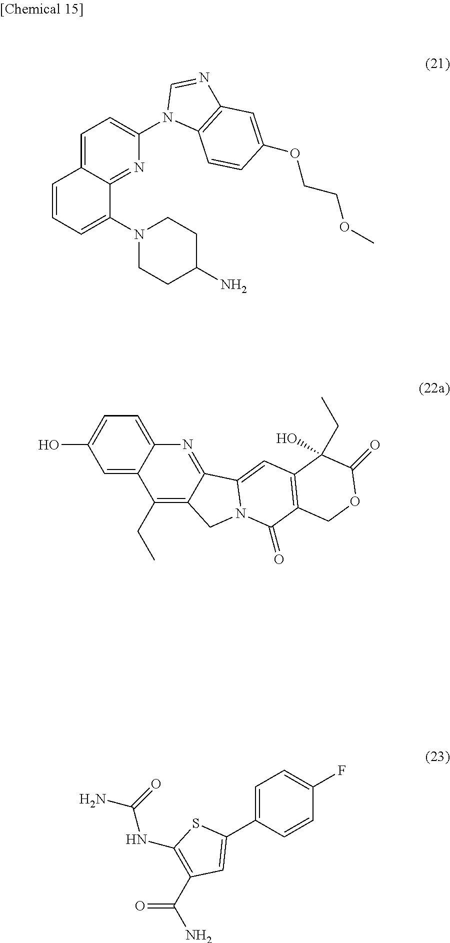 [SMILES: COCCOc1ccc2c(c1)ncn2-c1ccc2cccc(N3CCC(N)CC3)c2n1 ; CCc1c2c(nc3ccc(O)cc13)-c1cc3c(c(=O)n1C2)COC(=O)[C@]3(O)CC ; NC(=O)Nc1sc(-c2ccc(F)cc2)cc1C(N)=O]

### Example 2

Among the 23 kinds of compounds having a selective inhibitory action on SREBP-1, which had been identified in Example 1, a part of compounds (compounds (5), (6), (11), (16), (22a), and (23)) were found to reduce triglycerides in a culture solution of cultured hepatocytes.

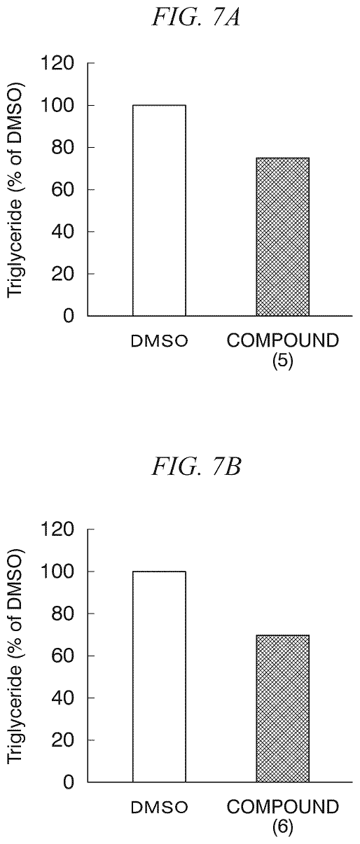

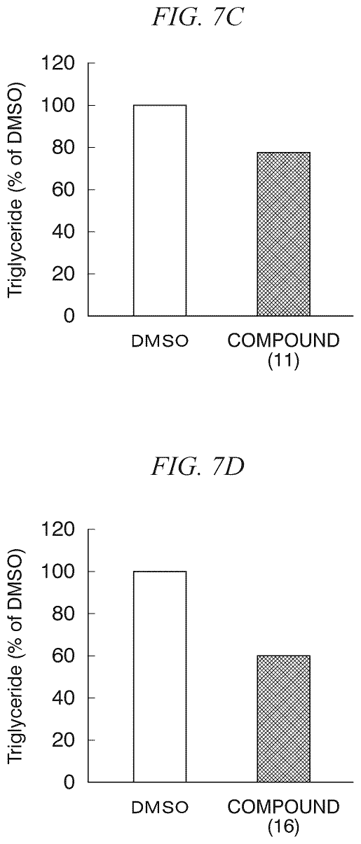

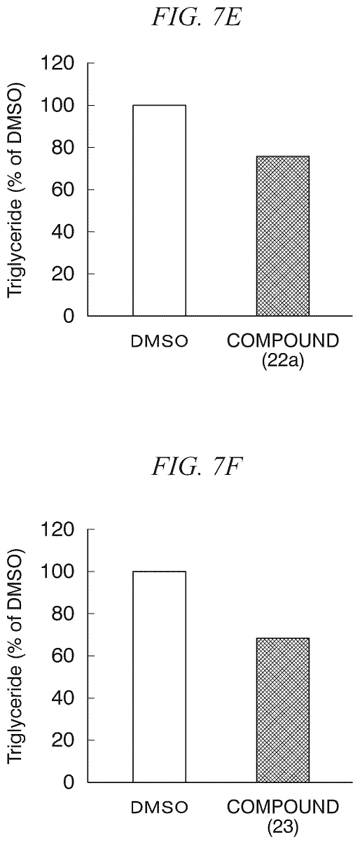

Specifically, first, H4IIEC3 cells (1×10⁵ cells/well) seeded in advance in a 48-well plate, which are a cell line of rat hepatocytes, were cultured for 1 day. Next, culture medium exchange was carried out so that a high glucose DMEM (serum-free) containing each compound (concentration in culture medium: 2 μM) or DMSO as a control was 200 μL/well, and then the cells were incubated for 24 hours. Next, the triglyceride concentration in the culture solution was measured using a Triglyceride E-Test WAKO (FUJIFILM Corporation). The results are shown in FIG. 7A to FIG. 7F. In FIG. 7A to FIG. 7F, the triglyceride concentration in each compound addition group was indicated as a relative percentage in a case where the triglyceride concentration in the DMSO addition group was set to 100%.

As shown in FIG. 7A to FIG. 7F, the action of reducing triglycerides was confirmed for all compounds.

From the facts described above, the above compounds can be expected to have an effect of reducing blood triglyceride levels and are expected to be applied to a therapeutic drug for hypertriglyceridemia.

### Example 3

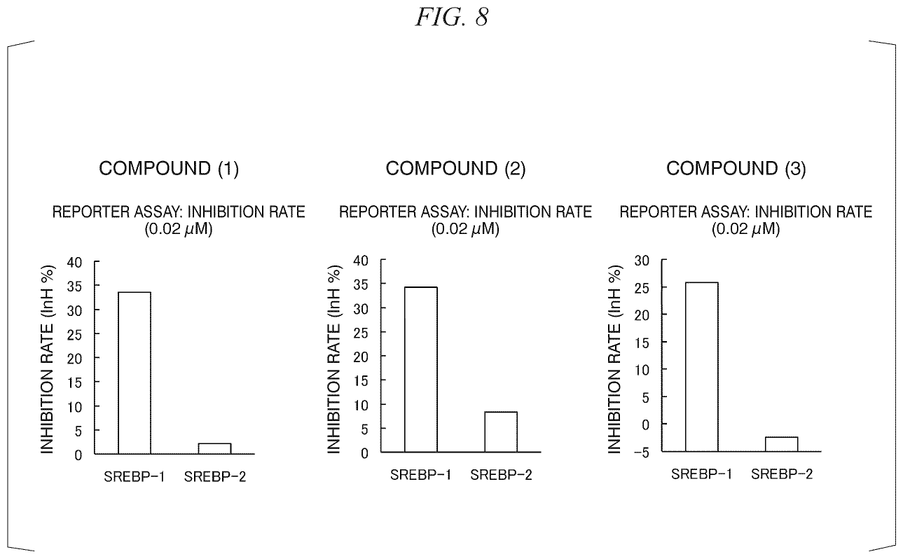

The compound (1) to the compound (3) were subjected to a reporter assay at a concentration in a culture medium of 0.02 μM according to the same method as in Example 1. The results are shown in FIG. 8.

The inventors of the present invention have confirmed that in the EPA which is an existing SREBP-1 inhibitor, a concentration at which the inhibitory action on SREBP-1 is exhibited is about 20 μM. In contrast, as shown in FIG. 8, the compound (1) to the compound (3) exhibits inhibitory action on SREBP-1 at a concentration of 0.02 μM, which revealed that these compounds have an inhibitory action stronger than that of EPA by about 1,000 times.

### [Industrial Applicability]

According to the SREBP-1 inhibitor according to the present embodiment, it is possible to provide an SREBP-1 inhibitor that specifically acts on SREBP-1. The pharmaceutical composition for treating hypertriglyceridemia according to the present embodiment contains the SREBP-1 inhibitor, and thus it is expected to be effective in the treatment of hypertriglyceridemia.

### SEQUENCE LISTING

PC35633_WA_Sequence Listing.xm

1. An SREBP-1 inhibitor that has inhibitory activity against SREBP-1 and has no inhibitory activity against SREBP-2, the SREBP-1 inhibitor comprising, as an active ingredient, any one or more compounds among compounds represented by Formulae (1) to (23).

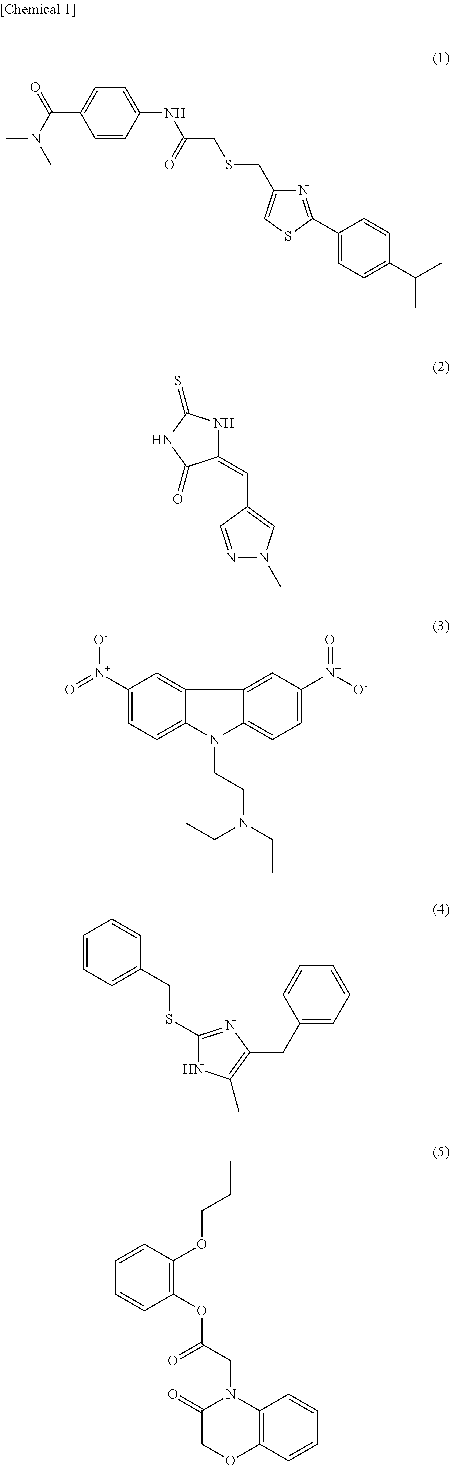 [SMILES: CC(C)c1ccc(-c2nc(CSCC(=O)Nc3ccc(C(=O)N(C)C)cc3)cs2)cc1 ; Cn1cc(/C=C2/NC(=S)NC2=O)cn1 ; CCN(CC)CCn1c2ccc([N+](=O)[O-])cc2c2cc([N+](=O)[O-])ccc21 ; Cc1[nH]c(SCc2ccccc2)nc1Cc1ccccc1 ; CCCOc1ccccc1OC(=O)CN1C(=O)COc2ccccc21]

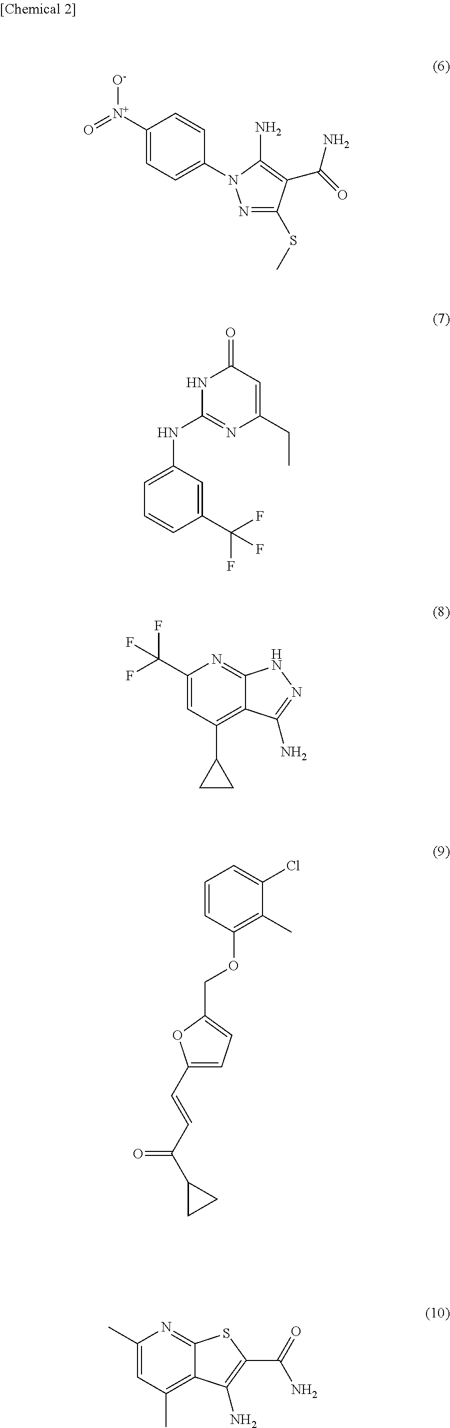

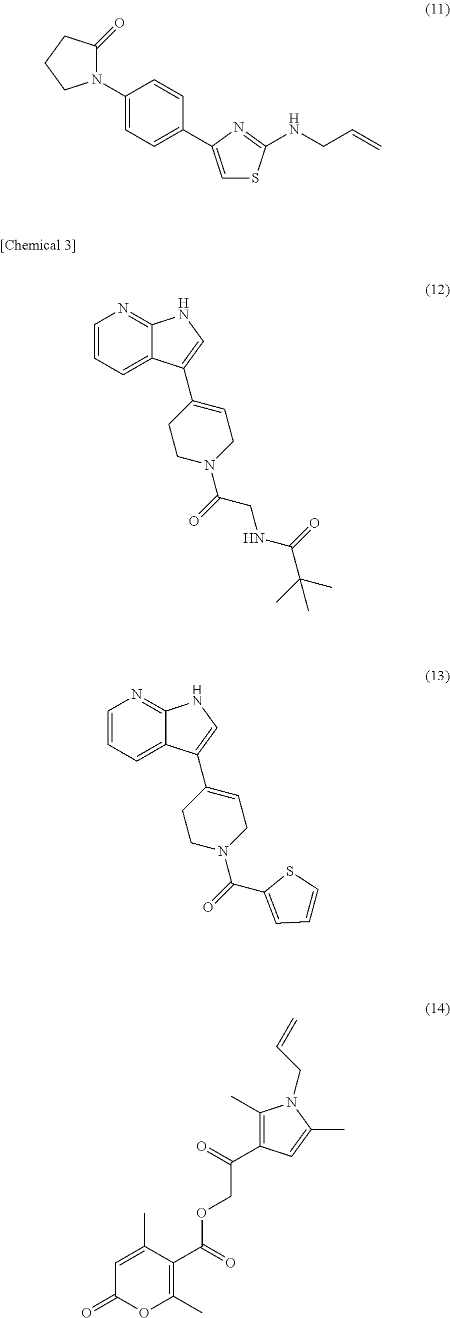 [SMILES: C=CCNc1nc(-c2ccc(N3CCCC3=O)cc2)cs1 ; CC(C)(C)C(=O)NCC(=O)N1CC=C(c2c[nH]c3ncccc23)CC1 ; O=C(c1cccs1)N1CC=C(c2c[nH]c3ncccc23)CC1 ; C=CCn1c(C)cc(C(=O)COC(=O)c2c(C)cc(=O)oc2C)c1C]

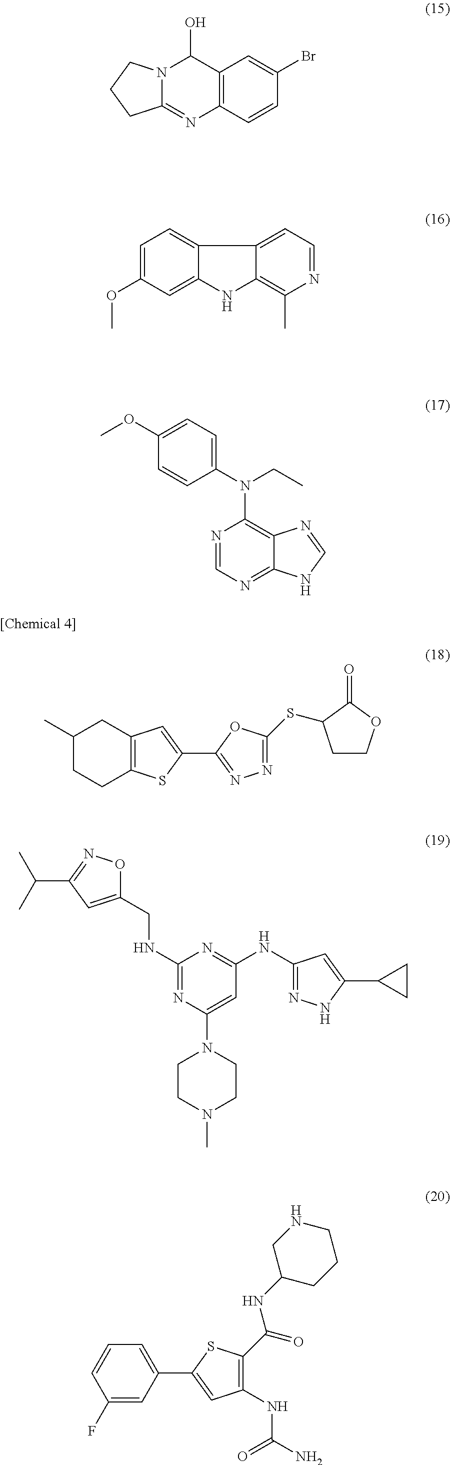 [SMILES: OC1c2cc(Br)ccc2N=C2CCCN21 ; COc1ccc2c(c1)[nH]c1c(C)nccc12 ; CCN(c1ccc(OC)cc1)c1ncnc2[nH]cnc12 ; CC1CCc2sc(-c3nnc(SC4CCOC4=O)o3)cc2C1 ; CC(C)c1cc(CNc2nc(Nc3cc(C4CC4)[nH]n3)cc(N3CCN(C)CC3)n2)on1 ; NC(=O)Nc1cc(-c2cccc(F)c2)sc1C(=O)NC1CCCNC1]

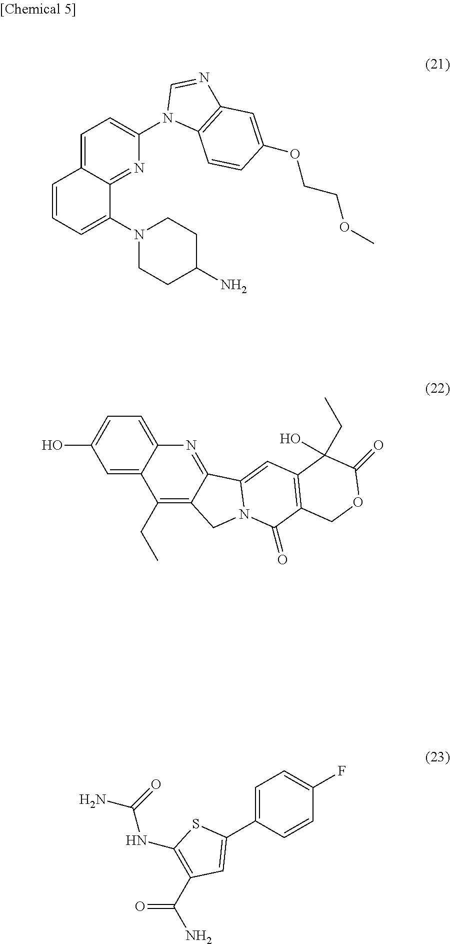 [SMILES: COCCOc1ccc2c(c1)ncn2-c1ccc2cccc(N3CCC(N)CC3)c2n1 ; CCc1c2c(nc3ccc(O)cc13)-c1cc3c(c(=O)n1C2)COC(=O)C3(O)CC ; NC(=O)Nc1sc(-c2ccc(F)cc2)cc1C(N)=O]

2. A pharmaceutical composition for treating hypertriglyceridemia, comprising:
the SREBP-1 inhibitor according to claim 1; and
a pharmaceutically acceptable carrier.
